(19) United States
(12) Patent Application Publication
(10) Pub. No.: US 2021/0022994 A1
(43) Pub. Date: Jan. 28, 2021

(54) SUSTAINED RELEASE TREPOSTINIL-COMPOUND MICROPARTICLE COMPOSITIONS

(71) Applicant: Lupin Holdings, B.V., Amsterdam (NL)

(72) Inventors: Okke Franssen, Ambt-Delden (NL); Evelyn Koekoek, Rotterdam (NL)

(21) Appl. No.: 16/936,383

(22) Filed: Jul. 22, 2020

Related U.S. Application Data
(60) Provisional application No. 62/877,302, filed on Jul. 22, 2019.

(51) Int. Cl. A61K 9/00; A61K 9/16; A61K 31/192; A61M 5/32

(52) U.S. Cl. CPC A61K 9/0019; A61K 9/16; A61M 5/329; A61K 9/1647; A61K 31/192

(57) ABSTRACT

Provided herein are new compositions comprising novel microparticles that are configured to provide a long acting release of one or more treprostinil compounds when administered to mammalian subjects. The microparticles of the invention are biocompatible and typically injectable through a needle or other injection system. The invention also provides methods of using such compositions, such as in the treatment of pulmonary arterial hypertension.

### CROSS-REFERENCE TO RELATED APPLICATIONS

This application claims the benefit of priority U.S. Provisional Patent Application 62/877,302, filed Jul. 22, 2019, the entirety of which is hereby incorporated by reference.

### FIELD OF THE INVENTION

This invention relates to microparticle compositions that are adapted to release treprostinil and related compounds in the body of mammalian subjects over extended periods. This invention also relates to the use of such compositions in therapeutic and other applications.

### BACKGROUND OF THE INVENTION

Prostacyclin (also known as prostaglandin 12 or PGI) is a lipid based, cardioprotective eicosanoid signaling molecule functioning in a wide variety of physiological systems and processes including inflammatory and immune response; cell growth regulation; control of blood pressure; and the modulation of regional blood flow to tissues. Prostacyclin is produced in the body by endothelial cells and is a powerful vasodilator as well as an inhibitor of platelet aggregation (an important component of the clot-forming process). Under healthy conditions, prostacyclin exists in equilibrium with other hormones influencing vasoconstriction.

Pulmonary arterial hypertension (PAH) is a progressive and debilitating condition that may lead to heart failure and death if not treated adequately. Five-year survival rates in PAH are on the order of 60-80%. Patients with PAH are found to have low levels of prostacyclin, which creates an unbalance in control of vasoconstriction and leads to a frequently life-threatening constriction of the pulmonary vasculature.

Patients suffering from low levels of prostacyclin and PAH are in need of a compensatory vasodilator, capable of addressing subpar levels of natural prostacyclin, to help dilate constricted blood vessels and improve the patients' ability to pump blood through the lungs. Natural prostacyclin, however, as a treatment mechanism is problematic as natural prostacyclin has been found to be unstable in solution and undergoes rapid degradation, making it very difficult to use in clinical applications. It is this characteristic that has led to the reported development of over 1,000 synthetic analogs of the molecule. Treprostinil is one such analog.

Treprostinil was first described in U.S. Pat. No. 4,306,075. Treprostinil (C₂₃H₃₄O₅) is a tricyclic benzidine prostacyclin analog also known as (1R,2R,3aS,9aS)-[[2,3,3a,4,9,9a-hexahydro-2-hydroxy-1-[(3S)-3-hydroxyoctyl]-1Hbenz[f]inden-5-yl]oxy]acetic acid. The compound has a molecular weight of about 390.52 g/mol. Like prostacyclin, treprostinil has demonstrated effectiveness in vasodilation, providing direct vasodilation of pulmonary and systemic arterial vascular beds, as well as inhibition of platelet activation. In addition, treprostinil inhibits smooth muscle cell proliferation. As a vasodilator, treprostinil is capable of reducing right and left ventricular afterload, increasing cardiac output, and increasing stroke volume; hence capable of improving dyspnea, fatigue, and exercise capacity.

Unlike natural prostacyclin, treprostinil has relatively good stability in neutral pH solutions and is stable at room temperature. This property in combination with its demonstrated efficacy has resulted in treprostinil being established as a potential component in PAH treatment modalities. However, while more stable than prostacyclin in solution, the fact that treprostinil has a half-life of only 2-4 hours in human plasma means that current treprostinil compound-based treatments require frequent administration to maintain therapeutically effective levels of the drug. Three such treprostinil compound PAH-treatments are presently marketed.

The first currently marketed product incorporating treprostinil as an active pharmaceutical ingredient (“API”) is formulated as an infusion, for subcutaneous or intravenous (IV) administration via a continuous infusion pump. This product (sold under the proprietary name REMODULIN™; US FDA approved in 2002) is provided in 20 mL vials containing 20, 50, 100, or 200 mg of treprostinil, with final active concentrations of 1 mg/mL, 2.5 mg/mL, 5 mg/mL, or 10 mg/mL. Infusion as a route of treprostinil administration is frequently associated with side effects, such as severe infusion site pain or reaction, and use of an infusion pump generally presents challenges in terms of patient compliance and patient monitoring, limiting or inhibiting successful use of this product.

The second currently marketed product incorporating treprostinil as an API is formulated as an inhaled aerosol, administered via a proprietary inhalation device 4 times per day (proprietary name TYVASO™; US FDA approved in 2009). The inhalation solution is provided in a 2.9 mL ampule containing 1.74 mg of treprostinil (0.6 mg/mL). It is dosed such that 6 μg of treprostinil are administered per “breath”, with a recommended target maintenance/max dose of 9 breaths, or 54 μg, per breathing session. Treprostinil administration via inhalation requires a disciplined regimen on the part of patients, presenting challenges in terms of treatment compliance.

The third currently marketed product incorporating a treprostinil composition (treprostinil diolamine), as an API is an oral tablet formulation, the tablet(s) being administered twice per day with approximately 12 hours between doses (proprietary name: ORENITRAM™; US FDA approved in 2013). Each tablet contains 0.125 mg, 0.25 mg, 1 mg, 2.5 mg, or 5 mg of active ingredient plus excipients. To achieve twice daily administration of this treprostinil compound use of an extended release tablet formulation is required. While this product offers greater convenience than the other two marketed treprostinil compound products, any treatment regimen requiring administration more than once a day is still less than optimal.

There appears to have been relatively few other reported attempts to develop treprostinil compound formulations, likely because typical approaches to drug formulation have failed or are expected to fail given the physiochemical properties of treprostinil compounds. There appears to be very little, if any, disclosure in the art relevant to micronized treprostinil compositions outside of nebulized inhalation formulations (comprising drug droplets of 2-10 μm, preferably less than about 5 μm in size) (see, e.g., US20120177693A1 and US20120216801A1). The lack of reported attempts to develop other micronized treprostinil compositions may be due to challenges with treprostinil, micronization generally, or both.

Micronization of drug particles has been demonstrated to be beneficial in developing formulations of pharmaceutical compounds with limited solubility, bioavailability challenges, and similar such factors. However, micronization has not consistently proven to be an effective pharmaceutical product development strategy. Conventional micronization (e.g., bead milling) may not be suitable for all drug substances (e.g., heat sensitive materials) and can be associated with low productivity, high cost, and product contamination. Additionally, such traditional micronization methods may result in compositions of particles with significant variation in size and/or shape, often resulting in less than optimal properties. Newer supercritical micronization methods have provided an alternative to such older methods; however, by relying on carbon dioxide, these methods also may be held back in some contexts by poor solvent power, high costs, and/or other concerns.

In general, reducing particle size also carries the potential risk of altering the morphology of the drug molecule, thereby potentially resulting in different polymorphs, amorphous APIs, or a mixture of crystalline and amorphous APIs. Micronized material also may be charged and may lead to segregation, clumping, and other possible physical instabilities. Microparticle compositions also can result in undesirable stimulation of the immune system. The results of micronization also may vary with the micronization method selected to produce the micronized drug particles.

Others have focused development efforts on pharmaceutical particles having size dimensions of less than about 1 micron in size. Such particles may be referred to as nanoparticles. International Patent Application WO2017192993 provides one example of a recent disclosure of an attempt to develop a new treprostinil composition using dried particles for inhalation that have a “pollen shape” and particle size width and length of 1-2 μm and depth of only 0.3-0.8 μm. Such ultra-small particles may not, however, be suitable for all treatment dosage forms, e.g., due to interaction of such small particles with phagocytic cells of the immune system.

### SUMMARY OF THE INVENTION

The present invention provides new compositions comprising microparticles that exhibit sustained release (or “long acting release”, sometimes abbreviated “LAR”) of one or more treprostinil compounds.

In one aspect the microparticles of the invention are characterizable as biocompatible, non-resorbable, injectable, and sustained release treprostinil compound particles. In another more particular aspect, the particles are also or alternatively characterizable as being biologically inert. In another aspect, the particles are characterizable as being resorbable as an alternative to being non-resorbable.

In any case the particles also are typically characterizable in having a relatively uniform size. E.g., most, a substantial majority, nearly all, or all of the particles can be composed of “monospheres” (spherical or spheroid particles having a substantially similar size composition).

In one aspect, the treprostinil composition is treprostinil or a pharmaceutically acceptable salt thereof. In other aspects, the treprostinil composition is a prodrug thereof, a hydrate thereof, a solvate thereof, or a polymorph thereof. In other aspects, the treprostinil compound is an analog or a derivative of treprostinil.

In one set of aspects, the particles of the composition comprise a carrier material, which makes up a substantial amount (e.g., at least about 20%—such as at least about 35%, at least about 50%, at least about 65%, at least about 75% or more) of the composition of the particle. The carrier material can be or can comprise a non-resorbable material, such as a bioceramic, as in the case of a BNIST particle composition (defined below). In other aspects, the carrier material can be or can comprise a resorbable material, as in the case of a BRIST particle composition (defined below). In a particular aspect, the invention provides BRIST particle compositions which comprise a resorbable polyester polymer carrier. In other aspects, the carrier material (or “carrier”) can comprise, can also or alternatively be primarily composed of, can be nearly entirely composed (e.g., at least about 80%, at least about 90%, at least about 95%, or at least 99% composed of), or is entirely composed of a copolymer.

In another set of aspects, the invention provides particle compositions wherein the particles have a relatively low amount of carrier material (i.e., less than about 20%, such as less than about 15%, less than about 12.5%, less than about 10%, less than about 7.5%, less than about 5%, or less than about 2.5% of the particle is made of a carrier material) or that lack any kind of carrier material. In one such aspect, the particles consist essentially or consist entirely of a treprostinil compound.

Such compositions are typically formulated with one or more diluents/solvents and often such compositions are formulated for needle injection, rather than for inhalation, infusion, or oral administration. Given the uniform distribution of particles in some of the compositions of the invention, the invention also provides kits and compositions comprising relatively small internal and/or external diameter needles or similar delivery devices for composition injection. Compositions in this and other contexts can comprise one or more excipients imparting various properties thereto.

The invention further provides methods of using such sustained release treprostinil compound compositions, such as in administration to a mammalian subject, wherein administration is once per day, once bi-daily (that is, every-other day), two or three times per week, once weekly, once monthly, or once over a longer period of weeks or months (e.g., once every 6 weeks, once every 2 months, once every three months, or once every 6 months), results in the release/delivery of an effective amount of the treprostinil composition over such an extended period of time. In some aspects, such methods are used to treat, enhance the treatment of, or prevent treprostinil-related conditions or to treat treprostinil-treatable diseases, such as PAH.

### BRIEF DESCRIPTION OF THE FIGURES

FIG. 1 is a comparison of particle size distribution of microsphere formulations fabricated using two different fabrication methods.

FIG. 2 is a schematic of a microsieve emulsification process which can be used to fabricate the particles of the invention.

FIG. 3 is a photographic image of particles of the invention within a dispersion in a 27 G needle.

### DETAILED DESCRIPTION OF THE INVENTION

### 1. Principles of Construction

To aid in the understanding of the disclosure provided herein, the following principles should be considered.

All references, including publications, patent applications, and patents, cited herein, including the patents and patent applications cited above, are hereby incorporated by reference to the same extent as if each reference were individually and specifically indicated to be incorporated by reference and were set forth in its entirety herein. Accordingly, the reader should review and consider such references in understanding the full content of this disclosure. For example, unless clearly contradicted by context or explicit statement, the disclosure of such documents relating to formulations, methods of production, and methods of use of compositions and devices can be combined with the teachings provided herein to provide additional useful compositions and applications. The he citation and incorporation of patent documents herein is limited to the technical disclosure of such patent documents and does not reflect on the validity, patentability, and/or enforceability of such documents.

All headings and sub-headings (e.g., “principles of construction”) are used herein for convenience only and should not be construed as limiting the invention in any way.

The use of the terms “a” and “an” and “the” and similar referents in the context of describing the invention are to be construed to cover both the singular and the plural, unless otherwise indicated herein or clearly contradicted by context.

The recitation of ranges of values in this document is intended to serve as a shorthand method of referring individually to each separate value falling within the range within an order of magnitude of the order of the recited range, including the endpoints. For example, a recited range of 1-2 should be interpreted as providing support for 1.0, 1.1, 1.2, 1.3, . . . 1.9, and 2.0 and a recited range of 10-20 is to be interpreted as providing support for 10, 11, 12, 13, . . . 19, and 20). Unless otherwise indicated, each such separate value is incorporated into the specification as if it were explicitly individually recited herein. All ranges provided herein include the end points of the provided range, unless the exclusion of such endpoints is clearly stated or clearly indicated, regardless of the terminology used to describe the range. Unless otherwise stated, all exact values provided herein are representative of corresponding approximate values and vice versa (e.g., all exact exemplary values provided with respect to a particular factor or measurement can be considered to also provide a corresponding approximate measurement, modified by “about,” where appropriate—e.g., disclosure of “about 10” is to be understood as supportive of 10 exactly).

Terms of approximation, such as “about” are used herein where measurements are understood to vary due to measurement issues or variability in populations, such as results of clinical studies. The scope of such terms will depend on the context of the element at issue and the understanding of those skilled in the art. In the absence of such guidance in the art, through relevant teachings or examples, the term “about” should be understood as meaning +/−10% of the indicated value(s). The term “approximately” is to be similarly interpreted.

Use of the term “or” herein is not meant to imply that alternatives are mutually exclusive unless clearly stated or clearly contradicted by context. Thus, in this disclosure, the use of “or” means “and/or” unless expressly stated or understood by one skilled in the art. In the context of any listed aspects of the invention written as depending on two or more other listed aspects, the use of “or” will be understood as referring back to any of the referenced aspects.

All methods described herein can be performed in any suitable order unless otherwise indicated herein or otherwise clearly contradicted by context or plausibility. Unless clearly indicated or contradicted by context or plausibility, the elements of a composition disclosed herein (e.g., a pharmaceutical formulation) can be formulated in any suitable manner and by any suitable method. Unless otherwise explicitly stated or clearly contradicted by context, any combination of the various elements, steps, components, and/or features of the aspects of the invention described herein, and all possible variations thereof, is to be considered encompassed by the invention.

Numerous examples and aspects are provided in this disclosure to better illuminate the invention. No example, particular aspect, or combination or pattern thereof is intended to pose a limitation on the scope of the invention unless otherwise indicated. No language in the specification should be construed as indicating any element is essential to the practice of the invention unless as much is explicitly stated. The breadth and scope of the present invention should not be limited by exemplary embodiments.

Although this document provides support for “means-plus-function” style interpretation of certain elements of the invention, both explicitly and implicitly, no element should be interpreted as limited to “means-plus-function” construction unless such intent is clearly indicated. In particular, the use of the terms “configured to” or “adapted to” are not intended to suggest a “means-plus-function” interpretation, but, rather, suggest that the relevant component, composition, device, or element has been configured to, designed to, selected to, or adapted to achieve a certain performance, characteristic, property, or the like using the principles described herein and/or that are generally known in the art.

Use of an element or component in the singular is to be understood as also providing simultaneous disclosure and support for a plurality of the element or component, if supported or otherwise understood to be possible. For example, discussion of a treprostinil compound in one context should be understood as providing support for one, two, or more treprostinil compounds unless clearly contradicted by context or an express contradictory statement. The converse also will be understood by those of ordinary skill in the art in reading this disclosure. In other words, the singular is intended to convey the plural and vice versa herein, unless otherwise stated or clearly contradicted by context.

The description herein of any aspect or embodiment of the invention using terms such as “comprising”, “having,” “including,” or “containing” with reference to an element, composition, or set of compositions or elements should be interpreted, whether explicitly stated or not, as simultaneously providing support for a similar aspect or embodiment of the invention that “consists of”, “consists essentially of,” “substantially comprises,” “materially comprises,” “predominately comprises,” “largely consists of,” and “substantially consists of” that particular element, unless otherwise stated or clearly contradicted by context (i.e., a composition described herein as comprising a particular element should be understood as also describing a composition consisting of that element, substantially comprising that element, predominately comprising that element, largely consisting of that element, and substantially consisting of that element, unless otherwise stated or clearly contradicted by context). Terms such as “including”, “containing”, and “having” should otherwise be interpreted openly herein, e.g., as meaning “including, but not limited to”, “including, without limitation”, or “comprising”, unless otherwise clearly contradicted.

The phrase “consists essentially of” and the descriptor “essentially” mean “do not materially affect the basic and novel characteristics” of the referenced step, element, method, or composition. The “basic and novel characteristics” will be clear to those of skill in the art from the description of the step, element, method, or composition provided herein, but in no event will be narrower than the scope of a corresponding description replaced with “substantially consists of” (defined below).

The phrase/term “substantially comprises” means that at least about 1% of a composition, population, or similar collection is or is made up of/by the referenced feature, species, or element, and typically means (and should be understood as providing support for) the relevant feature makes up or represents at least about 5%, at least about 10%, or even at least about 15% of the total amount of the e.g., composition or population.

“Materially comprises” means that at least about 20% of a composition, population, or similar collection is or is made up of/by the referenced feature, species, or element, and typically means (and should be understood as providing support for) the relevant feature makes up or represents at least about 25%, at least about 30%, or even at least about 40% of the total amount of the composition or number of the population.

“Predominately comprises” means accounting for more than one half (i.e., more than 50%) of a feature (e.g., a composition or a population of things). This amount and similar amounts used in respect of defined terms provided herein can be on a weight percent (weight/weight) basis, on a molecule/molecule basis, or other relevant basis used in the context of the relevant disclosure. For example, if a composition is described as “predominately comprising” element/species A, more than 50% of the composition on a molecular and/or weight basis will be made up of element/species A). Wherever this term is used it should be understood as simultaneously providing support for more than 60%, more than 70%, and more than 80% of the component or composition or collection being made up of the feature, species, or element at issue. The term “most” should also be construed similarly herein.

“Largely consists of” means that at least about 75% of the composition, population, or the like is or is made up of the referenced feature, species, or element at hand and should be understood as providing disclosure that at least 82.5%, at least 87.5%, at least 92.5%, and at least 97.5% of the composition, population, or the like is or is made up of/by the referenced feature, species, or element. Obviously, the remaining minority portion of the relevant composition, collection, and the like can be composed of other compounds, materials, or other relevant elements. The phrases “largely all” and “largely most” should be construed similarly.

“Substantially consists of” means at least about 90% of the composition, population, or the like is made up of the referenced feature, species, or element and should be understood as also providing disclosure that at least about 95%, at least about 99%, or at least about 99.9% of the composition, collection, etc., is made up of the relevant e.g., element or feature. “Nearly all” and “nearly entirely” should be construed similarly.

Changes to tense or presentation of phrases defined herein (e.g., using “comprises predominately” instead of “predominately comprises”) will not modify the meaning of the defined phrase, unless otherwise clearly indicated.

The embodiments described herein are described in sufficient detail to enable those skilled in the art to practice the invention. It is to be understood that other embodiments may be utilized without departing from the scope and spirit of the present invention. Therefore, the following detailed description should not be taken in a limiting sense, and the scope of the present invention is defined by the included claims, aspects and/or equivalents thereof.

Unless clearly indicated, the scope of any aspect or embodiment of the present invention is not limited to particular processes, compositions, or methodologies described, as these may vary. The terminology used in the description is for the purpose of describing particular versions or embodiments only; and is not intended to limit the scope of the present invention. Unless defined otherwise, all technical and scientific terms used herein have the same meanings as commonly understood by one of ordinary skill in the art. Any methods and materials similar or equivalent to those described herein can be used in the practice or testing of embodiments of the invention, the methods, devices, and materials described herein.

The description of the specific embodiments provided herein will reveal the general nature of the invention such that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. In general, it is to be understood that the phraseology or terminology herein is for the purpose of description and not of limitation, such that the terminology or phraseology of the present specification is to be interpreted by the skilled artisan in light of the teachings and guidance.

### 2. Overview and Introduction

This section provides an overview of exemplary and important aspects of the invention similar to, but in greater detail than, the Summary of the Invention provided above and provides definition to some of the key terms and concepts used in the remainder of this disclosure.

The present invention provides new compositions comprising microparticles that are adapted/configured for and exhibit sustained release (or “long acting release”) of one or more treprostinil compounds once administered into the body of a mammalian subject, such as a human.

The term “sustained release” (or “SR”) and the phrase “long acting release” (or “LAR”) means, in either case, release of an effective amount of a treprostinil compound, for a period of at least about one day (e.g., a period of about 1-100 days such as about 1-180 days).

The term “long acting release treprostinil compound microparticle (or particle)”, abbreviated “LARTCMP,” may also be utilized to describe a composition of microparticles that comprise treprostinil or another treprostinil compound and that have such a LAR characteristic.

The inventors have surprisingly discovered that suitable LARTCMPs can suitably deliver a LAR of treprostinil when delivered to the body of a mammalian host, such as a human PAH patient. The inventors have also surprising discovered that certain LARTCMP compositions can be prepared in a form that is adapted for formulation with one or more suitable diluents (often largely consisting of or substantially consisting of water) and injection delivery through relatively small needle/injection delivery systems. As such, the LARTCMP compositions of the invention can be advantageously used for medical applications, and offer numerous advantages over products known in the art. For example, these compositions can overcome the problems associated with more than once-a-day dosing administration and/or avoid inhalation of an aerosolized product or infusion delivery of treprostinil compounds.

As indicated, the compositions of the invention primarily relate to microparticles, which are generally understood as particles having an average size in all dimensions (e.g., an average diameter) of at least about 1 μm, but less than 1000 μm, and typically less than about 500 μm, less than about 300 μm, less than about 200 μm, or less than about 150, μm for example less than about 140, μm less than about 130 μm, less than about 120 μm, or less than about 110 μm. In most embodiments, the LARTCMPs will have a size of at least about 5 μm, and very often will have greater minimum sizes (e.g., at least about 8 μm, at least about 10 μm, or at least about 15 μm, for example at least about 20 μm, at least about 30 μm, at least about 40 μm, at least about 50 μm, at least about 60 μm, at least about 70 μm, or for example at least about 80 μm). Thus, typically most of the microparticles in a composition (e.g., at least about 65%, at least about 75%, at least about 80%, at least about 90%, at least about 95%, at least about 99% of the microparticles, or even about 100% of the microparticles in the composition) will have sizes that fall within a range of about 20-200 μm, typically about 30-180 μm, frequently about 50-150 μm, and still commonly about 75-125 μm, such as about 80-100 μm.

Phrases such as “particles having a diameter greater than x μm” herein refer to particles that will be retained by a filter having a cutoff of x μm and likewise phrases such as “particles having a diameter <y μm” refer to particles that are not retained by a filter having a cutoff of y μm. For example, the phrase “particles having a diameter of at least 20 μm” refers to particles that will be retained by a filter having a cutoff of 20 μm and the phrase “particles having a diameter of less than 200 μm” refers to particles refer to particles that are not retained by a filter having a cutoff of 200 μm. Where other particle size determination methods of equal or better reliability are available in the art, taking into account the composition to be analyzed and the possibility of nonuniformity of particle shapes therein, such other determinations can also or alternatively be used to determine particle sizes.

In another aspect, the invention provides compositions having a collection of microparticles that have sizes that fall within a limited range. In one such illustrative aspect, the invention provides compositions wherein the microparticles of the composition mostly, essentially entirely, or entirely fall within a range of just about 50 μm (from maximum diameter/dimension to minimum diameter/dimension). A composition wherein most, essentially all, or all of the microparticles have sizes of between 70 μm and 120 μm would, for example, satisfy such a condition. Still further aspects provide compositions wherein the size of microparticles mostly, essentially entirely, or entirely fall within a range of maximum-to-minimum sizes that is only about 40 μm or less in difference, e.g., about 30 μm or less, about 20 μm or less, or even about 20 μm or less in difference). Compositions defined by having microparticles of relatively uniform sizes can offer several advantages, e.g., ease of manufacture and relative uniformity of properties.

According to still other exemplary embodiments, at least 80% of the particles of the present invention have a maximum diameter of between about 5 μm and about 200 μm, such as between about 50 μm and about 125 μm (e.g., about 70-110 μm or about 80-100 μm). Again, this aligns well with the characterization of the particles of the inventive compositions being “microparticles.” The term “microparticle” herein means a particle having an average size of at least 1 micron in all dimensions, and typically at least 2.5 μm in all dimensions, and still very typically more than about 5 μm in all dimensions, such as at least about 7 μm in all dimensions, at least about 8 μm in all dimensions, or even at least about 9 μm in most dimensions or all dimensions (in at least most if not largely all, substantially all, or all of the particles in the composition). Microparticles herein typically have a maximum size of less than about 200 μm, and usually less than about 150 μm (e.g., about 120 μm or about 100 μm). The size and size distribution of microparticles according to certain embodiments is discussed in further detail elsewhere herein.

The term “microparticle” herein is typically used in a manner that is synonymous with the term LARTCMP. The term “particle” also may be used herein to refer to LARTCMPs with the understanding that such particles are microparticles having these size characteristics (e.g., typically having a size in all dimensions of at least about 5 μm).

In one aspect, LARTCMPs/microparticles of the invention are characterizable as biocompatible, injectable, and inert. In another more particular aspect, the particles are also characterizable as being also resorbable or are characterizable as being resorbable as an alternative to being inert. The microparticles can comprise a carrier. In some embodiments, the microparticles materially comprise, predominately comprise, or largely consist of a carrier material or a carrier system (comprising two or more carrier materials). In other embodiments, the microparticles comprise little or no carrier (e.g., in some embodiments the microparticles largely consist of, substantially consist of, consist essentially of, or consist entirely of a treprostinil compound).

In either case the particles also are typically characterizable in having a relatively uniform size and/or uniform shape. For example, most, a substantial majority, nearly all, or all of the particles can be composed of “monospheres” (spherical or spheroid particles having a substantially similar size composition).

The LARTCMP compositions of the invention can be subject to further formulation by combination with excipients, vehicles (e.g., solvents/diluents), or both in any suitable manner for any suitable application. The compositions of the invention are typically configured for/adapted for and formulated for delivery into the body via injection, such as by dispersing an effective amount of microparticles in a composition such as an aqueous liquid or gel, rather than administration via inhalation, infusion, or oral administration. The compositions of the invention can include additional excipients that impart different functions to the composition (e.g., anti-bacterial properties).

An exemplary aspect of the invention is embodied in a composition comprising biocompatible microparticles in an amount effective to prevent or treat a treprostinil-treatable disease or condition over a treatment period of at least one day in a significant number of patients suffering from or at risk of suffering from the disease or condition. In a particular exemplary aspect, at least about 90% of particles administered in such a method are monospheres and (a) have a maximum diameter of between about 5 μm and about 200 μm (e.g., about 8-160 μm, such as about 9-150 μm, about 20-140 μm, about 50-130 μm, or for example about 60-120 μm or 70 to about 120 μm), (b) comprise an amount of the treprostinil compound sufficient to provide for an effective daily release over the treatment period (e.g., about 1-60, about 1-90, or about 1-180 days), (c) optionally comprise an effective amount of carrier material that is associated with the treprostinil compound(s) when the particles are administered, and (d) are configured to release the effective daily release over a treatment period. Where present the carrier material can be an inert/non-resorbable material or a biodegradable and resorbable material.

The invention also provides methods of using such compositions for physiologic, prophylactic, or therapeutic applications, such as in the treatment of pulmonary arterial hypertension (PAH) or for a more general application, such as promoting vasodilation.

Additional disclosure relating to the overall scope of the invention is provided in the summary and in the Exemplary Aspects herein.

### 3. Treprostinil Compounds

The compositions of the invention can include any suitable treprostinil compound or combination of treprostinil compounds, alone or in combination with other APIs. A “treprostinil compound” can be any chemical compound that has sufficient functional and structural similarity to treprostinil to be useful in the methods described herein, such as in modifying a treprostinil-related condition in mammalian subjects, such as human patients. A number of treprostinil compounds suitable for use in the compositions and methods of this invention will be readily available to those of ordinary skill in the art either immediately or by application of routine experimentation and/or synthesis in accordance with the disclosure herein.

The treprostinil compound can also be a hydrate, solvate, salt, or polymorph of treprostinil or of any other treprostinil compound that is safe for administration to target mammalian subjects, typically humans, and that provides a desired physiological response upon such administration (e.g., a therapeutic effect with respect to a treprostinil-treatable condition). Thus, for example, a treprostinil compound can be a prodrug of treprostinil, an effective hydrate of treprostinil, a solvate of treprostinil, a polymorph of treprostinil, or a salt of treprostinil. According to certain exemplary embodiments, the treprostinil compound comprises, predominately comprises, largely consists of, substantially consists of, consists essentially of, or consists entirely of a prodrug of treprostinil. In accordance with further embodiments of the invention, the treprostinil compound comprises, predominately comprises, largely consists of, substantially consists of, consists essentially of, or consists entirely of a compound selected from an effective hydrate, solvate, polymorph, or salt of treprostinil.

In accordance with specific embodiments, the treprostinil compound comprises, predominately comprises, largely consists of, substantially consists of, consists essentially of, or consists entirely of treprostinil in its typical form.

The treprostinil compound also can comprise a mixture of two or more different treprostinil compounds selected from the group of treprostinil, a treprostinil derivative, or a treprostinil analog. As another example, a treprostinil compound can include a prodrug of treprostinil along with one or more of an effective hydrate, solvate, polymorph, or salt of treprostinil; an effective hydrate of treprostinil in combination with one or more of a solvate, polymorph, or salt of treprostinil; a solvate of treprostinil along with one or more of a polymorph or salt of treprostinil; or a polymorph of treprostinil along with a salt of treprostinil. Such combinations of two or more treprostinil compounds can be in any ratio to one another, such as for example a ratio of about 1:1, about 1:2, about 1:4, about 1:6, or about 1:8 or greater, such as about 1:10, about 1:15, about 1:20, about 1:25 or even higher, such as about 1:30, about 1:40, about 1:50, about 1:60, about 1:70, about 1:80, about 1:90, or about 1:100 or more, either compound capable of being the first or second component of the ratio. In compositions wherein the particles contain three or more treprostinil compounds, the ratio of the three such compounds can be any ratio to one another, such as for example about 1:1:1, about 1:2:1, about 1:1:2, about 2:1:1, about 1:3:1, about 1:1:3, about 3:1:1, about 1:4:1, about 1:1:4, about 4:1;1, about 1:5:1, about 1:1:5, about 5:1:1, about 1:6:1, about 1:1:6, about 6:1:1, about 1:7:1, about 1:1:7, about 7:1:1, about 1:8:1, about 1:1:8, about 8:1:1, about 1:9:1, about 1:1:9, about 9:1:1, about 1:10:1, about 1:1:10, about 10:1:1, or any ratio continuing the combination or ratios up to about 1:1:20, about 1:20:1, about 20:1:1, with, again, any such compound being capable of being the first, second, or third component of the ratio.

Salts of treprostinil compounds of the invention can typically be characterizable as “pharmaceutically acceptable”, as will be the nature of all components of the compositions of the invention in most aspects. “Pharmaceutically acceptable” means that the compound or composition being described is suitable for use in contact with the relevant tissues, circulation, or organs of the subjects/patients to receive the compound or composition. Pharmaceutically acceptable compositions typically lack any significant amount of toxicity (including carcinogenicity, genotoxicity, and mutagenicity), irritation, immunogenicity, allergenicity, and the like, and are commensurate with a reasonable benefit/risk ratio. Pharmaceutically acceptable salts suitable for the present invention are well known in the art and described in, for instance, Berge et al., “Pharmaceutical Salts,” J. Pharm. Sci., 66(1):1-19 (1977). For example, in one embodiment, a pharmaceutically acceptable salt of the present invention can be any acid addition salt, often a pharmaceutically acceptable acid addition salt, including, but not limited to, halogenic acid salts such as hydrobromic, hydrochloric, hydrofluoric and hydroiodic acid salt; an inorganic acid salt, such as, nitric, perchloric, sulfuric and phosphoric acid salt; an organic acid salt, such as, sulfonic acid salts (such as methanesulfonic, trifluoromethane sulfonic, ethanesulfonic, benzenesulfonic or p-toluenesulfonic), acetic, malic, fumaric, succinic, citric, benzonic gluconic, lactic, mandelic, mucic, pamoic, pantothenic, oxalic and maleic acid salts; or an amino acid salt such as aspartic or glutamic acid salt. The acid addition salt can be a mono- or di-acid addition salt, such as a di-hydrohalogic, di-sulfuric, di-phosphoric or di-organic acid salt. In all cases, the acid addition salt may be used as an achiral reagent which is not selected on the basis of any expected or known preference for the interaction with or precipitation of a specific optical isomer of the products of this disclosure.

Prodrugs are a class of compounds known in the art, and, accordingly, need only be briefly described herein. A prodrug will typically be characterizable as a functional derivative of a compound that is readily convertible to a parent compound in vivo. Prodrugs are often useful because, in some situations, they may be easier to administer than the parent compound. They may, for instance, be bioavailable by oral administration whereas the parent compound is not. The prodrug may also have enhanced solubility in pharmaceutical compositions over the parent compound. A prodrug may be converted into the parent drug by various mechanisms, including enzymatic processes and metabolic hydrolysis (as described in, e.g., Rautio et al., “The Expanding Role of Prodrugs in Contemporary Drug Design and Development,” Nature Reviews Drug Discovery 17:559-587 (2018); Clas et al., “Chemistry-enabled Drug Delivery (prodrugs): Recent Progress and Challenges,” Drug Discov. Today 19:79-87 (2014); Rautio, J. Prodrugs and Targeted Delivery, Wiley-VCH Verlag GmbH & Co. KGaA (2011); Stella, V. J. et al. Prodrugs: Challenges and Rewards Vol. 1-2, Springer & AAPS Press (2007); and for example in Rautio, J. et al., “Prodrugs: design and clinical applications,” Nat. Rev. Drug Discov. 7, 255-270 (2008)).

Solvates are similarly known in the art. Solvates typically are characterizable as complexes that are formed by combining the molecules or ions of a solvent with a compound. Solvates can also be characterized as being a complex that is obtained by the process of solvation where a solvent and one or more solute molecules reorganize into solvation complexes. Solvates typically include crystal forms containing either stoichiometric or nonstoichiometric amounts of a solvent.

Hydrates are another known class of compounds. Typically, a hydrate is a form of a compound that absorbs water molecules from its environment and includes them as part of its structure. In some instances, the hydrates of organic compounds are such that the water chemically reacts with the organic compound. In some instances, hydrates include a solvate where the incorporated solvent is water.

Those of ordinary skill will also be familiar with polymorphs. Polymorphs can be crystalline polymorphic forms or amorphous polymorphic forms of a compound, depending on the compound. Crystalline polymorphs have different arrangements and/or conformations of the molecules in crystal lattice. Amorphous polymorphs consist of disordered arrangements of molecules that do not possess a distinguishable crystal lattice.

The treprostinil compound of the compositions of the invention can also be a derivative of treprostinil. A derivative in this respect is a compound that comprises treprostinil or at least about 90% (e.g., at least about 95%) of the atoms of treprostinil in a corresponding arrangement to the arrangement of atoms in treprostinil, but that further includes one or more atoms, molecules, or groups that are associated with a treprostinil compound core through covalent bonds. Typically a derivative will comprise all or essentially all of the corresponding underivatized treprostinil compound structure, but which further comprises the addition of one or more atoms, groups, moieties, side chains, and the like which typically will impart modified functionality to the derivative compounds, while retaining at least most of the properties of treprostinil in terms of its ability to induce desired physiological, therapeutic, prophylactic or similar effects when administered.

The treprostinil compound also can be an analog of treprostinil. An analog in the context of this disclosure means a compound that is both similar structure and similar pharmacologic/physiologic function as treprostinil. Those of ordinary skill will typically be able to assess whether a compound should be considered an analog of treprostinil. Typically, an analog of a compound such as a treprostinil analog will comprise at least about 80%, at least about 90%, or at least about 95% of the atoms present in treprostinil in the same configuration. Also or alternatively, a compound can be considered an analog of treprostinil when it differs from treprostinil by 1, 2, 3, 4, 5, 6, 7, 8, 9, 10, 11, or 12 modifications (differences) which can be considered to be selected from substitutions, additions, and/or deletions of atoms in treprostinil. Typically, such modifications will retain core components of the structure of treprostinil required for function of that compound and the analog, such as the central three ring core structure of the compound. For example, in one aspect the treprostinil analog possesses a tricyclic benzidine prostanoid structure and exhibits vasodilator properties such that the analog is capable of detectably and ideally therapeutically reducing pulmonary vasoconstriction while also exhibiting acceptable levels of effects, ideally minimal detectable effects, on systemic blood pressure.

The LARTCMPs (microparticles) of the invention typically will include an amount of one or more treprostinil compounds effective for the intended use of the composition. An effective amount in this respect can be any amount suitable for causing one or more intended effects, typically effects in a mammalian subject, and commonly in a human patient, or that has been determined to likely cause such an effect, based on effectiveness in a similar population of similar subjects. Because of the variability of subjects and patients, the effective amount often will be an amount that has been demonstrated to be effective in the relevant context in a significant proportion of a population of subjects or patients (e.g., as determined by one, two, or more well controlled and adequate clinical studies).

In aspects a particle of the inventive composition can comprise an amount of treprostinil compound (which may sometimes be described as a “drug load”) in a range of about 1 weight percent (“wt %”) to about 80 wt % or about 1 wt % to about 100 wt %. E.g., compositions of the invention can comprise treprostinil in a concentration ranging from about 1 wt %, about 5 wt %, about 10 wt %, about 15 wt %, about 20 wt %, about 25 wt %, about 30 wt %, about 35 wt %, about 40 wt %, about 45 wt %, or about 50 wt %, for example about 55 wt %, about 60 wt %, about 65 wt %, about 70 wt %, about 75 wt %, about 77.5 wt % or about 80 wt %. In aspects, as will be discussed elsewhere herein, particles of the invention can sometimes include higher concentrations of treprostinil compound such as at least about 90 wt %, at least about 95 wt %, at least about 98 wt %, or at least about 99 wt % of a treprostinil compound, and in some cases can be entirely composed of a treprostinil compound, e.g., about 100 wt % (e.g., about 100.0 wt %) of a treprostinil compound. Often times the term percent (%) herein is used in actual reference to wt %, particularly with respect to the content of compositions.

The precise amount of treprostinil compound in a particle can vary in accordance with the nature of the particle and the intended concentration of the treprostinil compound therein (the drug load). The amount of treprostinil compound in the composition comprising the particles can vary with the intended application, and will reflect an amount of treprostinil that is effective for the intended use in view of the intended effect of the composition when administered, the dosing regimen, and the release properties of the particles (e.g., amount of composition administered, frequency of administration, and rate of release of the treprostinil compound when administered). In certain aspects, the amount of treprostinil compound present in a composition, such as a single dose for administration, can fall within a range of from about 0.1 mg-about 2000 mg, e.g., about 0.1 mg-about 2100 mg, such as about 10 mg-about 1800 mg, as in about 50 mg-about 1500 mg, e.g., about 100 mg-about 1400 mg, about 100 mg-about 1250 mg, about 100 mg-about 1000 mg, about 200 mg-about 1200 mg, about 250 mg-about 1250 mg, about 250 mg-about 1000 mg, about 250 mg-about 750 mg, about 400 mg-about 1200 mg, about 400 mg-about 800 mg, or about 500 mg-about 1000 mg.

### 4. Microparticle Characteristics

The primary component of most of the compositions and methods of this invention are microparticles comprising an effective amount of one or more treprostinil compounds and that are configured to provide a sustained release of such treprostinil compounds following administration to the body (in other words, LARTCMPs). As noted elsewhere, LARTCMPs will typically have a size of less than about 0.25 mm (e.g., less than about 200 μm (μm)), but in this invention typically will mean particles having a size of less than about 0.15 mm or very often less than about 0.1 mm in size.

In some cases, e.g., due to manufacturing processes, a microparticle composition is better characterized in that at least about a certain amount of particles in the composition are microparticles or are microparticles of a certain size (e.g., at least about 95%, at least about 98%, at least about 99%, or even at least about 99.5% of the particles in the composition are microparticles meeting the desired particle range limits, such as about 5-150 μm, about 7-130 μm, about 9-110 μm, or about 10-90 μm). The particles of the invention can fall within a lower end of this range, however in some embodiments can more typically fall within the upper end of these ranges, such as, e.g., at least about a certain amount of particles in the composition, such as at least about 95%, at least about 98%, at least about 99%, or even at least about 99.5% of the particles in the composition are microparticles of a size falling between about 50-about 130 μm, such as between about 60-about 120 μm, between about 70-about 110 μm, or for example between about 80-about 100 μm. In one exemplary aspect, the invention provides microparticle compositions that largely consist or substantially consist of microparticles that fall between about 6 μm and about 95 μm, such as between about 7 μm-about 90 μm. In a more common aspect, the invention provides microparticle compositions that largely consist or substantially consist of microparticles that fall between about 50 and 130 μm, such as between about 70-about 110 μm.

### A. Particle Composition Characteristics

In addition to having the size characteristics described above and comprising an effective amount of one or more treprostinil compounds, the particles of the invention typically can be characterized by possessing and/or exhibiting a number of additional advantageous properties.

The first such property is that the particles will be biocompatible, meaning that the particles will not be deleterious to living tissue that the particles contact when administered. The assessment of biocompatibility is understood in the art. Biocompatibility will typically mean that the particles lack toxic components or are essentially free from toxic, allergenic, and/or immunogenic components (e.g., less than about 0.01 wt %, typically less than about 0.001 wt %, or less than about 0.0001 wt % of the particle is composed of such toxic, allergenic, carcinogenic, or immunogenic materials).

The particles of the invention also will typically be injectable, as will be further discussed below. This means that the particles can be formulated in a dispersion by contact with one or more suitable diluents/solvents and the dispersion formulation delivered through an injection system, such as a needle delivery system, to the tissues of a mammalian subject. It is important to note that although this represents a key aspect of the invention, that does not mean that the particles must be delivered in such a manner, as they can alternatively be delivered by implantation or other delivery procedure.

The injectability of the particles typically relates to the maximum size of the particles, the size distribution of the particles, and the shape of the particles, all of which are factors relating to the size of any particle aggregates that can form in compositions of the invention, e.g., aqueous dispersions of particles. Large aggregates can cause needle and other injection systems to clog and fail. Such principles are discussed herein.

Another feature of the particles is that they provide a sustained release (extended release, long acting release) of the treprostinil compound(s) that are in the particles. The sustained release effect of the particles can be attributed to the presence of a carrier system, the configuration of the particles, or both. As will be discussed elsewhere herein, particles of the invention can largely consist of, substantially consist of, consist essentially of, or consist entirely of treprostinil, but still provide a sustained release of the compound.

As already stated elsewhere herein a “sustained release” or LAR means a release of at least one day, and typically over a period of about 1 to about 180 days. The release refers to release of an effective amount of the treprostinil compound. As discussed and described elsewhere herein an “effective amount” can vary with the context of the intended use, but generally means an amount of the composition that is sufficient for providing the desired effect in the intended recipient or, more commonly, that is capable of providing the desired effect in a significant proportion of a population of similar subjects (e.g., as determined by identification of a statistically significant effect in one or more well controlled, adequate clinical studies of the composition). The period of extended release can be, according to exemplary aspects, at least about 1 week, at least about 2 weeks, at least about 1 month, about 2, 3, or even about 6 months, for example about 20-about 30 weeks, such as about 22-about 26 weeks).

As stated in the preceding paragraph, the particles of the invention can be characterized as comprising a carrier system, having a low amount of carrier, or lacking any carrier system components. Particles that have carrier systems can be further divided on the basis of whether such carrier materials are resorbable or inert. Inert carriers are intended to remain indefinitely in the body and are not typically subject to resorption in the body of most subjects. Resorbable carrier materials on the other hand will resorb into the body after a period, although such period can often have a significant duration (e.g., about 6 months or more, about 8 months or more, about 14 months or more, about 20 months or more, or even about 24 months or more on average) and may vary from individual to individual. It is also possible for a particle to include a mixture of such different carrier materials and for a composition to comprise particles of all four types.

### I. Summary of Possible Particle Composition Characteristics

As indicated from the preceding paragraphs, there are a number of different characteristics that are typically present in particles of the invention and the particles may be separated into four different classes based on their carrier material characteristics. An overview of such characteristics is provided below as Table 1.

TABLE 1
Overview of Particle Composition Characteristics

| Common Microparticle | Variable Carrier |
|---|---|
| Characteristics | Characteristics |
| All or substantially all microparticles | Particles (especially resorbable |
| are under 200 μm in size, and | particles): |
| typically all or substantially all | can comprise no carrier or little |
| particles are under 120 μm in size | carrier or |
| Biocompatible | can include an effective amount |
| | of one or more carrier materials |
| Particles are injectable (have suitable | Where particles include a carrier - |
| maximum size, size distribution, | Particles can comprise a resorbable |
| shape, or a combination thereof) | carrier or |
| Comprise an effective amount of | Particles can comprise a non- |
| one or more treprostinil compounds | resorbable carrier |
| Provide a sustained release of the | Where particles include a carrier - |
| treprostinil compound(s) (e.g., an | carrier materials may be bioactive |
| effective daily dose is released for | or carrier materials may be inert |
| about 1-about 180 days) | |

As will be exemplified below, the compositions of the invention can include particles that exhibit various combinations of the variable carrier characteristics, but usually will comprise most if not all of the common characteristics, and these characteristics can be combined with other variable characteristics concerning, e.g., the size of the particles, the shape of the particles, or the presence of other contents such as excipients or other APIs. Both the common characteristics and variable carrier characteristics of the particles of the invention are described in more detail below.

### II. Carrier Materials/Systems (Generally)

A “carrier material” or “carrier” in this disclosure means any material contained in a particle of the invention or that otherwise makes up a part of a particle of the invention, along with the treprostinil compound(s) and any additional API(s) in the particle, which is not classified as an API or an excipient.

An “excipient” is a component of the composition in which the particles are contained other than diluents/base solvent(s) (e.g., water). In one aspect, an excipient is a compound, molecule, or composition which, at least in some contexts, is bound to the surface of a particle or makes up part of the particle. In other aspects, an excipient is a compound or molecule that also or alternatively makes up part of a solvent system in which the LARTCMPs are dispersed. In either case, an excipient can, if present in an effective amount, which can typically be the case, exhibit a functional property in the context of the composition (e.g., a preservative function, a surfactant function, etc.). In one aspect, an excipient can be an adjuvant, meaning a composition that detectably aids in the function of the treprostinil compound and/or other APIs contained in the LARTCMP. In some cases, excipients that are bound to or mixed with carrier particles (e.g., in a dry composition) will in other contexts be released from or otherwise become more disassociated from the particle (e.g., when a mixture of excipient and particle are reconstituted).

Excipients are often variously described in the art by terms such as stabilizers, diluents, bacterio- or fungi-static agents and the like, antioxidants, buffers, suspending or thickening agents, or any combination thereof. Compositions of the invention can include any suitable excipient or combination of excipients. Non-limiting examples of excipients that can be included in compositions can include a stabilizer, a preservative, a bactericide, fungicide, or other agent to maintain sterility, one or more chemicals to assist in dissolving dried particle material, a buffer, a salt (e.g. sodium chloride so as to form a saline solution), one or more surfactants (e.g., one or more non-ionic surfactants); or one or more preservatives or any combination of any two or more thereof. In one aspect, the excipients comprise a surfactant. In another aspect, a composition of the invention also or alternatively includes one or more suitable polymeric or hydrophobic excipients. As exemplified elsewhere herein, one aspect of the invention is embodied in dried microparticle compositions, which may or may not comprise one or more such excipients, which are then added to liquid formulations (reconstituted), typically prior to use, which liquid formulations also or alternatively can include one or more excipients.

According to some embodiments, a particle composition can comprise one or more excipients, most or all of which can, upon reconstitution, separate from the particle such that part of the total amount of excipient(s) or adjuvant(s) present can be bound to the particle in suspension and part or all of the total amount of excipient(s) or adjuvant(s) originally present in the particle composition becomes a solvent in a solution formed in reconstitution or suspension/dispersion of the microparticles. According to further embodiments, one or more excipient(s) or adjuvant(s) of the formulation also or alternatively can be part of a liquid composition diluent. Such embodiments are exemplified elsewhere herein.

Typically, a carrier material can detectably change one or more performance characteristics of the particles. Such an effect is typically directly on the particles, rather than on other composition components. In one aspect, the presence of the carrier material detectably changes the sustained release profile of the particle, typically by detectably increasing the period of release of an effective amount of the treprostinil compound.

In one aspect the carrier material, can be characterized by having an average resorption time in human subjects of less than about 2 years, for example less than about 18 months, or less than about 15 months. According typical embodiments, the carrier material can be resorbed, on average in human subjects, within less than about 12 months, for example less than about 11 months, less than about 10 months, less than about 10 months, less than about 9 months, less than about 8 months, less than about 7 months, or for example a carrier material can be resorbed within about 6 months (on average in human subjects—in some cases the carrier material is resorbed in at least about 65%, at least about 80%, at least about 90%, or at least about 95% of human subjects within about 12 months, within about 10 months, within about 9 months, within about 7 months, within about 6 months, or any of the other referenced resorption times). Carrier materials also can include “filler” materials that are used to increase the size of the microparticle, where a certain larger size is desired. Carrier materials can also provide other functional properties, such as detectability, tissue targeting, enhancement of stability, adjuvant effects, or similar effects/properties. A “carrier system” in this disclosure means a collection of two or more carriers, with or without other “filler” materials, though this does not require that such two or more carriers necessarily have to be in contact or function together. As described elsewhere herein, the particles can alternatively be associated with a single composition carrier as opposed to two or more carriers. In other words, a single particle, some particles of a composition, or all particles of a composition can comprise a single-material carrier or a multiple-material carrier. In a related sense, a full population of particles within the composition can be comprised of particles comprising a single material, particles comprising multiple materials, or a mixture of particles some of which comprising carriers of a single material and some of which comprising carriers of multiple materials.

The compositions of the invention can include any suitable amount of a carrier material or carrier system. As noted, in some respects, compositions of the invention can include very little carrier material (e.g., about 15 wt % or less, about 10 wt % or less, about 5 wt % or less, about 3 wt % or less, about 2 wt % or less, about 1 wt % or less, about 0. wt5% or less, or even about 0.1 wt % or less). In other aspects, the particle can materially comprise, predominately comprise, or even largely consist of a carrier material or carrier system. Thus, in exemplary embodiments, particles of the invention are provided that comprise about 20-about 100 wt %, such as about 20-about 80 wt %, e.g., about 25-about 75 wt % or about 35-about 70 wt % of the particle.

The carrier material or carrier system typically is present in an amount capable of facilitating the release of (is configured to release) a treprostinil compound loaded onto, in, and/or with the carrier material over the course of a treatment period. Where present, carrier material will be typically present in an amount and/or configured to detectably modify (and usually sustain) the release of the treprostinil compound during an intended treatment period.

According to some embodiments, the composition of the present invention can comprise particles wherein at least about 30% of the composition of the particles can be composed of the carrier. For example, at least about 30%, at least about 35%, at least about 40%, at least about 45%, at least about 50%, or more, for example at least about 55%, at least about 60%, at least about 65%, at least about 70%, at least about 75%, at least about 80% or even more, for example at least approximately 85%, at least approximately 90%, or at least approximately 95% of the composition of the particles is composed of the carrier or a carrier system. In such cases, the remainder of the particle composition can comprise in some part one or more treprostinil compounds, optionally in addition to one or more excipients. For example, wherein at least about 30% of the composition of the particles can be composed of the carrier, up to about 70% of the composition of the particles can be comprised of one or more treprostinil compounds. Exemplary embodiments can include particles that, for example, are composed of about 30% carrier and about 70% treprostinil, about 35% carrier and about 65% treprostinil, about 40% carrier and about 60% treprostinil, about 45% carrier and about 55% treprostinil, about 50% carrier and about 50% treprostinil, about 55% carrier and about 45% treprostinil, about 60% carrier and about 40% treprostinil, about 65% carrier and about 35% treprostinil, about 70% carrier and about 30% treprostinil, about 75% carrier and about 25% treprostinil, about 80% carrier and about 20% treprostinil, about 85% carrier and about 15% treprostinil, about 90% carrier and about 10% treprostinil, about 95% carrier and about 5% treprostinil, or greater than about 95% carrier and less than about 5% treprostinil or other treprostinil compound.

Typically, the carrier material will be composed of a suitable polymer or copolymer, such as a resorbable polymer or copolymer, which are described in the following section of this disclosure, or a ceramic material (which often may be characterized in the art as a “bioceramic”), which can be resorbable or non-resorbable and/or inert or bioactive. Thus, for example, carrier material can be, e.g., a non-resorbable bioceramic, a resorbable bioceramic, or a mixture of the two.

Bioceramics that can be used as carriers can include fully crystalline, partially crystalline, or non-crystalline bioceramics. Crystalline bioceramics can be of a single crystalline material or alternatively a polycrystalline material. Bioceramics can include bioactive glass (“bioglass”), calcium carbonate (also referred to as coral or natural coral), calcium sulfate, biologically derived calcium phosphates such as those derived from bone, coral or algae) or synthetic calcium phosphate compounds. According to a specific embodiment, the bioceramic can be resorbable, crystalline bioceramic tricalcium phosphate (TCP). According to alternative embodiments, the bioceramic can be a calcium phosphate selected from the group of biphasic calcium phosphate (BCP), octacalcium phosphate (OCP), a di-calcium phosphate (DCP), or a monocalcium phosphate (MCP). According to some embodiments, the bioceramic can be hydroxyapatite (HA) (a bioactive and bioresorbable calcium phosphate, the bioresorbability being variable in its rate and extent).

Numerous additional types of carrier materials will be characterized and further described below, as will mixtures of such materials. The appropriate carrier material can be selectable based on the application of the active agent, e.g. the present active or actives and the intended therapeutic target of the composition or formulation can require a carrier material if present to have specific resorbability, bioactivity, or similar characteristic which can determine the most appropriate carrier material to utilize in the composition or formulation.

### III. Resorbable Carriers/BRIST Particles

In one aspect the invention provides particles that comprise, substantially comprise, materially comprise, predominately comprise, or even largely consist of one or more biodegradable and resorbable (“bioresorbable”) carriers (the terms “resorbable”, “resorption” and the like are used to signify that the referenced material is both broken down and then resorbed by the tissues of the body over a period of time). Such biocompatible, resorbable, injectable, sustained release treprostinil compound particles can be referred to as “BRIST” particles. BRIST particles represent one important aspect of the invention.

Although the carrier in such a particle can be characterized as being resorbable in such aspects, the resorbability of the carrier material is typically such that the particles remain detectable in the body for a sustained period after administration. In one exemplary embodiment, at least about 75% of the particles in the composition, e.g., at least about 80% of the particles in the composition, such as at least about 90% the particles of the composition are resorbable within 6 months of administration, at least about 9 months of administration, at least about 12 months of administration, at least about 18 months of administration, at least about 21 months of administration, or about 24 months of administration or longer (e.g., about 18-30 months or about 20-26 months) (e.g., as determined through in vivo studies). According to embodiments, at least about 95% or at least about 99% or at least about 99.5% of the particles are resorbed within about 2.5 years or about 2 years of administration. In another facet, also or alternatively at least about 50%, at least about 70%, at least about 80%, at least about 90%, or at least about 95% of the particles do not resorb within about 3 months, within about 6 months, within about 9 months, within about 12 months, or within about 15 months from administration. However, in other aspects, shorter resorption time particles can be provided (e.g., particles in which the particle is resorbed within 3 months or less, 2 months or less, 1 month or less, or 1 week or less). In other aspects, a proportion of the particles can be resorbed and/or retained (not resorbed) for such periods of time, such as at least about 50% of the initial particles, at least about 70% of the initial particles, at least about 80% of the initial particles, at least about 90% of the initial particles, or at least about 95% of the particles are resorbed and/or retained for one or more of the indicated periods provided earlier in this paragraph.

In other certain embodiments, the carrier material when present in the composition can be resorbable within about 1 year or longer. For example, at least about 75% of the resorbable carrier material, such as about 80%, about 85%, about 90%, about 91%, about 92%, about 93%, about 94%, about 95%, about 96% or more, for example about 97%, about 98%, about 99%, about 99.5%, or more, such as about 100% of the resorbable carrier material can, upon injection, be resorbed within the physiological environment of the recipient within about 2 years, such as within about 18 months, within about 12 months, for example within about 11 months, within about 10 months, within about 9 months, within about 8 months, or is resorbed within about 7 months from the time of administration, for example within about 6 months.

According to alternative embodiments, the carrier material can exhibit a delayed resorption, such that the carrier material or particle is detectable in the recipient (at least in a large proportion of the initially administered particles, such as at least about 25%, at least about 50%, or at least about 75% of the initially administered particles) for about 1 year or more, for example about 1.5 years, about 2 years, about 2.5 years, or about 3 years. In a typical embodiment, the carrier material can exhibit an resorption time of less than about 1 year, such that less than about 25%, such as less than about 20%, less than about 15%, less than about 10%, less than about 5%, less than about 4%, less than about 3%, less than about 2%, less than about 1%, or less than about 0.9%, less than about 0.8%, less than about 0.7%, less than about 0.6%, less than about 0.5%, less than about 0.4%, less than about 0.3%, less than about 0.2%, or even less than about 0.1% of the initially administered particles are undetectable after approximately 1 year or less, such as for example after about 12 months, after about 11 months, after about 10 months, after about 9 months, after about 8 months, after about 7 months, after about 6 months, after about 5 months, after about 4 months, after about 3 months, after about 2 months, or even after about 1 month.

In aspects, no detectible amount of particles remains in a patient approximately six months, approximately five months, approximately four months, approximately three months, approximately two months, or approximately one month post-administration of a composition to patients. In aspects, there is no statistically significant occurrence of detection of particles remaining in a population of recipients of a composition described herein about six months, about five months, about four months, about three months, about two months, or about one month post-administration.

The resorbable carrier material can be formed from or comprise a resorbable polymer (e.g., the carrier material can materially comprise or predominately comprise a resorbable polymer). The polymer can be a single polymer, a copolymer, or a mixture of polymer and/or copolymer materials. Typically, the polymer will be a synthetic polymer.

As already indicated, a polymer material serving as a carrier material can be a mixture, or matrix of polymers and/or copolymers. The polymeric matrix material can be selectively chosen based on such criteria as biocompatibility, biodegradability (e.g. resorption profile), mechanical properties, cosmetic appearance, interface properties, the selection of which being further driven by the particular application of the active in the formulation(s) of the present invention.

According to embodiments, the composition of the carrier material of particles in the present invention can be comprised of at least about 1% of a biodegradable resorbable polymer or copolymer, for example at least about 1%, at least about 2%, at least about 5%, at least about 10%, at least about 15%, at least about 20%, or at least about 25% of the carrier material of particles can be comprised of a biodegradable resorbable polymer or copolymer. In another facet, at least approximately 30%, at least approximately 35%, at least approximately 40%, at least approximately 45%, or at least approximately 50% of the carrier material is a biodegradable resorbable polymer material (which can be a single polymer, a copolymer, or a mix of polymers or copolymers). In other aspects, the composition primarily comprises such a polymer material, e.g., at least approximately 55%, at least approximately 60%, at least approximately 65%, at least approximately 70% or greater of the carrier material of particles can be comprised of a biodegradable resorbable polymer, such as at least about 75%, at least about 80%, at least about 80%, at least about 85%, at least about 90%, at least about 92.5%, at least about 95%, at least about 97.5%, at least about 98%, at least about 98.5%, at least about 99%, at least about 99.5%, or at least about 99.9% of the carrier material of particles can be comprised of a biodegradable polymer. The invention provides compositions in which about 1-about 90%, about 2-about 80%, about 5%-about 75%, or about 10% to about 65%, e.g., about 12.5% to about 50% of the carrier material is composed of one or more biodegradable resorbable polymers.

In other aspects, the composition primarily comprises such a copolymer material, e.g., at least approximately 55%, at least approximately 60%, at least approximately 65%, at least approximately 70% or greater of the carrier material of particles can be comprised of a biodegradable resorbable copolymer, such as at least about 75%, at least about 80%, at least about 80%, at least about 85%, at least about 90%, at least about 92.5%, at least about 95%, at least about 97.5%, at least about 98%, at least about 98.5%, at least about 99%, at least about 99.5%, or at least about 99.9% of the carrier material of particles can be comprised of a biodegradable copolymer. The invention provides compositions in which about 1-about 90%, about 2-about 80%, about 5%-about 75%, or about 10% to about 65%, e.g., about 12.5% to about 50% of the carrier material is composed of one or more biodegradable resorbable copolymers.

In one embodiment the resorbable carrier is composed of material(s), such as a biodegradable resorbable polymer, copolymers, or polymer mixture, which primarily, essentially completely, or completely undergoes surface erosion biodegradation. In another embodiment, the resorbable polymer carrier is composed of a material or mix of materials, such as one or more polymers that primarily, essentially entirely, or entirely undergoes bulk erosion. In still another aspect, the biodegradable resorbable carrier material exhibits both types of erosion.

In one aspect, the particles can comprise a polyanhydride polymer and/or one or more polymers which undergo surface erosion, which, for example, can be a sebacic acid (SA), p-carboxyphenoxyproane (CPP) copolymer, or which can comprise SA or CPP as individual polymers. A SA-CPP copolymer can comprise a combination of SA and CPP in any ratio sufficient to confer the desired, targeted properties of the LARTCMP formulation (and such compositions can be validated for possessing a target range of SA and CPP monomers). In aspects, the polymer can be or comprise a polyanhydrids-co-imide which can undergo surface erosion. In further aspects, the biodegradable polymer can be a poly(ortho)ester, which can be designed so as to undergo surface erosion, and can optionally further be characterizable as a type IV poly(ortho)ester or a self-catalyzed poly(ortho)ester. Examples of such polymers are described in, e.g., Ng. et al., “Synthesis and erosion studies of self-catalyzed poly(ortho ester)s”, Macromolecules, 30 (4), pp. 770-772 (1997).

In aspects, the biodegradable polymer can comprise one or more resorbable polyester polymer (“RPP”) materials. In further embodiments, the RPP material can be composed of a polyglycolide (PGA), a polylactic acid (PLA), a polycaprolactone (PCL), polyhydroxybutyrate (PHB), a copolymer of any two or more thereof, or a mixture of any two or more thereof. According to yet further embodiments, an RPP material making up a carrier can comprise a poly(lactic-co-glycolic) acid (PLGA). Such a PLGA can primarily comprise the single crystalline, optically pure isomer poly-L-lactic acid (PLLA), the single crystalline, optically pure isomer poly-D-lactic acid (PDLA), or can be amorphous, having atactic ordered polymer chains, comprising a mixture of PLLA and PDLA, sometimes referred to as PDLLA. According to one embodiment, a typical polymer of the present invention can be a copolymer of DL-lactide or a copolymer of DL-lactide and glycolide.

In one aspect, the RPP particles comprise, substantially comprise, materially comprise, largely consist of, consist essentially of, substantially consist of, or consist entirely of PLA that is selected from poly-L-lactic acid (PLLA) (crystalline) or poly-D-lactic acid (PDLA) (crystalline), or a mixture thereof (PLLA and PDLA (homopolymer, crystalline) or PDLLA (copolymer, amorphous)). In one aspect, the RPP particles comprise, substantially comprise, materially comprise, consist largely of, substantially consist of, consist essentially of, or consist entirely of PLLA.

In one aspect, the RPP particles comprise, substantially comprise, materially comprise, largely consist of, consist essentially of, substantially consist of, or consist entirely of a copolymer of DL-lactide (a PDL copolymer) or a copolymer of DL-lactide and glycolide (a PDLG copolymer). Such copolymers can exist in varying molar ratios, such as for example a 50/50 ratio, 55/45 ratio, 60/40 ratio, 65/35 ratio, 70/30 ratio, 75/25 ratio, 80/20 ratio, 85/15 ratio, 90/10 ratio, 95/5 ratio, or similar ratio, the ratio selected based on the product profile being targeted, such but no limited to, for example, the product active release characteristics. In certain embodiments, the copolymer selected is an acid-terminated copolymer. Examples of such polymeric materials are provided elsewhere herein.

In one aspect, the invention provides a microparticle composition, where the microparticle comprises an RPP copolymers material. As is understood in the art and discussed elsewhere herein, the constituents of RPP copolymers can be modified to change the properties of the RPP material. E.g., PGA has no methyl side groups and has a higher crystalline structure than PLA; whereas the methyl side groups of PLA make it more hydrophobic than PGA. Therefore, PLGA copolymers richer in PLA are less hydrophilic, absorbing less water and as a result degrading more slowly in the body. “Tuning” of the, e.g., hydrophilicity, hydrolytic group interaction, crystallinity, and volume-to-surface ratio of the PLGA allows a user to generate such a molecule with a modified degradation times ranging from, e.g., less than about one month to more than about six months. In one aspect, the polymer is a material, such as an RPP, that has been adjusted by incorporation of a specific ratio of materials or other modification to achieve one or more of such properties, typically also meaning that the material is validated against a certain standard that is the target of such “tuning.”

According to embodiments, the RPP particles comprise, largely consist of, or substantially consist of and/or consist essentially of or consist entirely of PLLA particles that are composed of poly-L-lactic acid, a poly-D-lactic acid, or a mixture thereof.

According to embodiments, the RPP particles comprise, largely consist of, or substantially consist of and/or consist of, or consist entirely of a DL-lactide copolymer or a copolymer of a DL-lactide and glycolide. Such copolymers may or may not be acid-terminated.

The molecular mass of a polymer carrier material, such as a RPP, e.g., a PLA polymer, e.g. a DL-lactide copolymer, DL-lactide and glycolide copolymer, or a PLLA polymer, that makes up most if not substantially all of most, substantially all, or all of the particles of a composition of the invention, calculated by viscometry (i.e., the viscosity average molecular weight (“Mv”)), is typically about 5,000-about 650,000 Daltons. According to more particular embodiments, the My of the primary or only polymeric carrier material of the LARTCMPs of the composition is about 10,000-about 250,000 Daltons, such as about 80,000-120,000 Daltons, e.g., about 90,000-about 110,000 Daltons, and in some cases about 100,000 Daltons.

According to embodiments, the inherent viscosity of a carrier material polymer, such as a RPP polymer, more particularly such as a PLA polymer, or even still more specifically a PLLA polymer, that makes up most if not largely all, substantially all, or all of the particles of a composition of the invention is also or alternatively between about 0.1 dL/g and about 4.0 dL/g. In more specific embodiments RPP carrier material that has an inherent viscosity of about 0.1-about 2.0 dL/g is incorporated into the LARTCMP. In still a more precise aspect, the RPP carrier material has an inherent viscosity of about 0.50-about 1.10 dL/g, e.g., about 1 dL/g.

In a further aspect, particles of the invention are provided comprising an RPP carrier, as exemplified by PLA, which can also or alternatively be a chiral polymer. In a more specific aspect, the polymer can be an optically active, levorotary polymer. In exemplary aspects, the polymer can be PLLA with a specific rotation of between about −150 and −160 cm²/g, commonly abbreviated as simply degrees)(°.

In an embodiment, particles of the invention are provided comprising an RPP carrier, as exemplified by a DL-lactide copolymer or a DL-lactide and glycolide copolymer having a specific rotation of about 0 degrees, for example between approximately −5 and approximately 5 degrees, such as between about −4 and about 4 degrees, between about −3 and about 3 degrees, between about −2 and about 2 degrees, and such as having a specific rotation of between about −1 and about 1 degree.

Carrier materials incorporated into microparticles of the inventive compositions are sometimes characterized on the basis of the melting point of the material, which reflects properties of the material that are in some contexts important or desirable to control. In general, carrier materials can have any suitable melting point. In one exemplary aspect, the carrier material, such as an RPP, such as a PLLA, of some, most, substantially all, or all of the particles of a composition can have a melting point of between about 170° C. and about 200° C., such as 175-195° C., and in a more specific embodiment between 177 and 192° C. (e.g., 178.0-190.1° C.). Also or alternatively, wherein the heat of fusion (also referred to as enthalpy of fusion) of 100% crystalline PLLA is about 90 J/g, the RPP of the particles in the present invention, such as PLA in most, having a mixture of PLLA and PDLA polymers, can have a heat of fusion ranging between about 50 J/g and about 100 J/g, such as between 85 J/g and 90 J/g.

Materials that meet the various above-described features of carrier polymers are known or can be produced using methods known in the art with undo experimentation. For example, a PLLA product having the low residual monomer, low residual solvent, high melting temperature, and high heat fusion characteristics of specific aspects provided above, and/or a PDL copolymer or a PDLG copolymer as described above, are currently commercially available from Corbion (Gorinchem, The Netherlands) and Evonik Nutrition & Care GmbH (Essen, Germany).

RPP carrier materials can biodegrade in vivo through nonspecific hydrolysis and can release monomeric constituent(s) that are naturally endogenous and/or readily metabolized in a mammalian recipient, such as lactic acid, glycolic acid, or both compounds.

According to certain embodiments, the polymer carrier material comprises a PLGA and the properties of PLGA are tailored to key elements of product performance such as administration mode or also or alternatively drug release rate by changing the block ratios of constituents and the molecular weight of the polymer. Such modifications provide for the ability to select the diffusion rate of the active and the degradation rate of the polymer which should match the desired release period. According to certain embodiments, the polymer can be selected such that the release rate reduction over time, a typical characteristic for diffusion-controlled release formulations, is compensated for by the degradation related release rate, which increases over time. In such embodiments, polymer mixtures can comprise any ratio of the two isomers PLLA and PDLA so as to confer the desired characteristics of the formulation, such as drug release rates and material resorption rates. In one aspect, such materials are selected and/or validated for having such properties.

In alternative embodiments, the carrier material used in the microparticles of a composition will primarily, essentially, or entirely be composed of a material that has a resorption rate that is measurably less than that of PLLA or PDLA, such that the carrier material resorbs in less than about 1 year, such as less than about 10 months, such as less than about 8, less than about 7 or less than about 6 months in most, nearly all, essentially all, or all detectable cases Thus, although acceptable in some aspects, in many aspects, the polymers of the present invention are not PLLA and/or PDLA based materials (or materials with similar resorption properties). In exemplary embodiments copolymers of DL-lactide or a copolymer of DL-lactide and glycolide can be used as a primary or only carrier material in most, essentially all, or all microparticles of a composition. Such copolymers may or may not be acid-terminated. According to some embodiments, copolymers can be those with a degradation profile of 1 year or less, such as about 9 months or less, or about 6 months or less. Specific exemplary polymers having one or more of the described characteristics of carrier material which can be used in the microparticles of a composition are currently commercially available as PDLGA5002A, an acid-terminated copolymer of DL-lactide and glycolide in a 50/50 molar ratio; PDLG7507, a copolymer of DL-lactide and glycolide in a 75/25 molar ratio; and PDL02, a copolymer of DL-lactide (Corbion; Gorinchem, The Netherlands).

PLA polymers that can be used as carrier material in particles of the invention can range from amorphous glassy polymers to semi-crystalline and high crystalline polymers. It is known art to utilize different technologies to modify or enhance the mechanical properties of PLA. As non-limiting examples, annealing, adding nucleating agents, forming composites with fibers or nanoparticles, chain extending, and introducing cross-linking structures are all ways of modifying the mechanical properties of PLA that can be employed in the context of particles of the invention.

According to certain embodiments, the carrier material can comprise a PLGA polymer, wherein the PLGA copolymer has a monomer ratio between about 50 and about 100. For example, a PLGA copolymer according to a specific embodiment can have a monomer ratio of about 1-about 100 (i.e., about ratio 50:50 to about ratio 100:1, typically meaning the polymer comprises a ratio of such materials ranging from about 1-50 monomers of lactic acid to every 1-50 monomers of glycolic acid to about 100 monomers of lactic acid to every 1 monomer of glycolic acid or vice versa (e.g., about 1:1)). In more particular aspects, the invention provides microparticles comprising a PLGA copolymer that has a monomer ratio of about 1-50, about 60-40, about 70-30, about 80-200, about 90-10, or about 100-1 (e.g., about 50, about 55, about 60, about 65, about 70, about 75, about 80, about 85, about 90, about 95, or about 100).

According to certain embodiments, the carrier material can comprise an RPP material having a molecular weight of between about 5 kDA to about 200 kDa. For example, the molecular weight of a RPP utilized as a carrier material in certain embodiments of the present invention can be about 5 kDa, about 10 kDa, about 20 kDa, about 30 kDa, about 40 kDa, about 50 kDa, about 60 kDa, about 70 kDa, about 80 kDa, about 90 kDa, or about 100 kDa, such as for example about 110 kDa, about 120 kDa, about 130 kDa, about 140 kDa, about 150 kDa, about 160 kDa or for example about 170 kDa, such as having a molecular weight of about 180 kDa, 185 kDa, 190 kDa, 195 kDa, or about 200 kDa.

RPP carrier material of particles provided herein also, can, in embodiments, be homopolymers, e.g., a polymer consisting of identical monomer units. In aspects, the RPP carrier material can be an aliphatic resorbable polyester homopolymer (an ARPH). Other resorbable polymers comprising polyester monomer components mixed with other components, and having the other properties associated with the various types of particles of the invention described herein (e.g., substantially uniform size, shape, and/or both) could be also or alternatively incorporated in the compositions and systems and/or used in the methods of the invention.

RPP materials typically are synthetic (produced through chemical synthesis starting with either synthetic or natural materials, or both). Thus, such materials avoid inclusion of (i.e., are free from) bovine and other animal-derived materials. A number of RPP products are derived from plant materials. In one context, RPP particles can be derived from such natural sources. In another context the RPP particles can be also or alternatively produced through chemical synthesis using synthetic starting materials.

A number of RPP compositions are known, and several have been used to varying degrees for aesthetic modification of humans. Such RPP materials can make up the most, if not all, of the RPP component of most if not all of the particles of any composition provided herein. In one aspect, the RPP material includes, substantially consists of, consists essentially of, or consists of (at least within the level of detection) a polyglycolide (a “PGA block”). Polyglycolides are known in the art and discussed elsewhere herein. In another aspect, the RPP material of the particles similarly is or includes a polycaprolactone (a “PCL block”). Such materials are also known in the art. In still another aspect, the RPP composition similarly is or includes a polyhydroxybutyrate (a “PHB block”), which is a material that is understood in the art. In still another aspect the RPP is or similarly includes a polylactic acid (or a “PLA”). PLAs are described and exemplified elsewhere herein and also are known in the art. The RPP particles also can include mixtures of two, three, or more of such materials and/or can include copolymers formed from two or more of such materials. In some aspects, the RPP particles will substantially consist or consist of only one type of RPP material. In some aspects, RPP particles will substantially consist or consist of only RPP material. In some aspects, RPP particles will substantially consist or consist of RPP material that consists of only one type of RPP (e.g., the particles will consist entirely of a PLA, such as PLLA). In other aspects, the herein described blocks or monomers can form an RPP composed from (randomly) altered monomeric units.

Those of ordinary skill in the art will recognize that often particle compositions will not be completely pure due to normal aspects of the production process. Thus, e.g., RPP particle compositions can contain some amount of residual solvents, residual monomers, or both. Residual solvents are leftover solvents from the manufacturing process, which are not removed, but that are considered to be present at an acceptable level. Residual monomers (e.g., lactic acid in the case of PLA) are monomers of an RPP that were not polymerized during the polymer production process, which usually also are present at a level considered to be suitable. The RPP(s) used in the particles will according to some aspects have be associated with less than about 1%, such as less than 0.1%, less than 0.05%, or less than 0.01% residual solvents (e.g., less than 0.005% or less than 0.001% residual solvents). The RPP used in the particles will also or alternatively be associated with less than about 2%, less than about 1%, less than about 0.5%, or less than about 0.1% residual monomer (e.g., lactic acid in the case of a PLA carrier material) (such as less than about 0.05%, less than about 0.025%, or less than about 0.01% residual monomer).

In another aspect, the carrier material can be or can comprise a naturally occurring material or can be derived from a naturally occurring material, in some cases with limited modification such that most of the natural properties of the natural material are maintained or are substantially maintained. In other aspects, the material can be a synthetic version of such a material. In one such aspect, the carrier material of the particles of the present can comprise a proteinaceous material. Such a proteinaceous material can include such materials as keratin, elastin, bone, collagen, extracellular matrix material, or the like. Proteinaceous material carrier materials can be advantageous in certain aspects as such materials typically are biocompatible, biodegradable, and also can be highly “tunable”, that is, can be subject to known modifications to attain specific material characteristics. In one aspect, the proteinaceous material is a material that has been modified prior to incorporation into the particle to achieve such “tuning” and/or the material has been analyzed/validated and subject to selection and rejection and/or purification in order to achieve such tuning prior to incorporation into the particle.

A naturally occurring carrier material, such as a proteinaceous carrier, can be present in any suitable amount, which can correspond to the amounts of polymer materials described elsewhere herein. Thus, for example, a carrier material that is composed of a natural material, such as a protein, according to certain embodiments of the present invention can be present in an amount such that at least about 1% of the carrier material can be comprised of one or more proteinaceous materials. According to some embodiments, at least about 1% of the carrier material is a proteinaceous material, such as at least about 2%, at least about 4%, at least about 6%, at least about 10%, or more, for example at least about 15%, at least about 20%, at least about 25%, at least about 30%, at least about 35%, or at least about 50% of the carrier material is proteinaceous. In other embodiments, the carrier material of the LARTCMP predominately comprises a proteinaceous material. In one aspect, approximately 55%, at least approximately 60%, at least approximately 65%, at least approximately 70%, or at least approximately 75% of the carrier material is a proteinaceous material. In still other aspects, at least approximately 80%, at least approximately 85%, at least approximately 90%, at least approximately 95%, at least approximately 99%, at least approximately 99.5%, or at least approximately 99.9% of the carrier material is composed of one or more proteinaceous materials.

In aspects, a naturally occurring carrier material or derivative thereof is a material that is considered biologically well-defined, as is the case with bone or dermal collagen materials. Purified proteins and protein mixtures also would have such characteristics. Other naturally occurring proteinaceous materials can include extracellular matrix components, which can be subject to various levels of purification or characterization/uniformity.

The carrier can be comprised of two or more proteinaceous materials, such two or more proteinaceous materials that can be combined in any ratio providing the targeted characteristics of the composition (e.g. drug release rate or level of bioactivity).

Also or alternatively, a proteinaceous material can be used in combination with a non-proteinaceous material as the base for the carrier material in specific embodiments of the particles of the invention. In such scenarios, such materials can be utilized in any ratio in relation to one another conferring the targeted characteristics of the carrier material (e.g. drug release characteristics, bioresorption rate, or level of bioreactivity). Additional combinations of material carriers are discussed further below.

In some aspects, the resorbable carrier material is a resorbable ceramic (ceramic materials that are suitable for administration into the body of subjects are often alternatively referred to as “bioceramics” in the art). Any suitable biodegradable and resorbable bioceramic material can be used as a carrier material in such contexts. Biodegradable/resorbable bioceramics include aluminum-calcium-phosphate (ALCAP) ceramics (e.g., Al—Ca—P oxides); glass fibers and their composites; corals (e.g., Corraline—a material derived from any of various red algae of the family Corallinaceae whose fronds are covered with calcareous deposits); calcium sulfates; calcium carbonates; zinc-calcium phosphorous oxide (ZCAP) ceramics; zinc-sulfate-calcium-phosphate oxide (ZSCAP) ceramics; and ferric-calcium-phosphorous oxide (FECAP) ceramics. Biodegradable/resorbable bioceramics can also or alternatively comprise or be calcium phosphate ceramics, including calcium phosphate salts and resorbable apatite ceramics (e.g., ceramics defined by the formula M₁₀(XO₄)6Z₂, where M=Ba, Ca, Ce, K, Na, Pb, Sr and Y; X═As, P, and Si; and Z═F, Cl, O, OH and H₂O, which can have substitution at certain sites, such as the Z sites, according to principles known in the art). Yet another type of resorbable material are silica-calcium phosphate nanocomposites. Tricalcium phosphate materials are often resorbable and have been frequently used as carriers in various devices and can be incorporated into particles of the invention as a carrier material. These and other ceramics described herein can be derivatized (or “doped”—e.g., addition of OH—, F—, NaF—, CaO—, MgO—, ZnO—, or SiO₂— groups), sintered, or similarly modified, resulting in different properties such as greater density, stability, or both.

Another resorbable material that can be used as a carrier in some embodiments is hydroxyapatite (e.g., Ca₁₀(PO₄)₆(OH)₂). A hydroxyapatite carrier can be defined by its density and/or C/P ratio. An exemplary hydroxyapatite material can have, for example, a set Ca/P ratio, e.g., about 10/6 (e.g., about 8-12: about 4-8) and/or a density of about 3-3.5 g/ml, such as about 3.2 g/ml.

According to specific embodiments, the carrier material utilized in the particles of the present invention can comprise an amount of a resorbable bioceramic. According to further embodiments, at least about 1% of the bioceramic carrier material can be a resorbable bioceramic, for example at least about 1%, at least about 2%, at least about 5%, at least about 10%, at least about 15%, at least about 20% or at least about 25% of the carrier material utilized comprises a resorbable bioceramic, such as at least about 30%, at least about 35%, at least about 40%, at least about 45%, or at least about 50% of the material is a resorbable bioceramic material. In other aspects, the carrier material of the LARTCMP predominately comprises a bioresorbable carrier. According to embodiments, at least about 55%, at least about 60% or more, such as at least approximately 65%, at least approximately 75%, at least approximately 85%, at least approximately 90%, at least approximately 95%, at least approximately 97.5%, at least approximately 99%, or even about 100% (or 100%) of the carrier material utilized in the particles of the present invention can comprise a resorbable bioceramic.

### IV. Non-Resorbable Carriers

As indicated above, in some contexts LARTCMPs can comprise a non-resorbable (“NR”) carrier, often which is an inert and non-resorbable carrier material or carrier system, or a carrier material or carrier system that contains ether type of such materials. Particles that are composed of biocompatible injectable non-resorbable sustaining-release treprostinil compound materials can be referred to as “BNIST particles.”

A non-resorbable material can be any material that will, on average, undergo less than about 10%, less than about 5%, less than about 2%, or less than about 1% resorption over a period of at least 10 years, but typically for a longer period such as at least 15 years, at least about 20 years, at least about 25 years, at least about 30 years, or longer. Typically, such materials will usually remain substantially intact following administration to the body throughout the remainder of the subject's lifespan, unless removed. The term “inert” means that the material elicits no detectable tissue response or interaction. Not all non-bioresorbable materials are inert and bioactive carrier materials that are non-bioresorbable or resorbable are discussed below (though specific examples of such materials are also exemplified in this section and the preceding section).

BNIST microparticles can comprise any suitable non-resorbable carrier material or combination of two or more materials of such characteristics. The carrier particles can be, for example, formed from an alumina, typically an aluminum oxide (e.g., a compound of or comprising Al₂O₃), such as a sapphire alumina, or can comprise such a material (e.g., the carrier can materially comprise such an alumina material). In another aspect, the carrier material can be or can comprise a zirconia based ceramic material. Examples of such materials include yttrium stabilized tetragonal polycrystalline zirconia, zirconia/alumina composites, zirconia toughened alumina, and ceria and/or magnesia doped zirconia. According to other embodiments, the NR carrier can be or can comprise a NR carbon material. For example, the NR carrier can be formed of a vitreous carbon material; a vapor-deposited carbon material (e.g., an ultra-low-temperature isotropic carbon, or ULTI); or a pyrolytic carbon material (e.g., a low-temperature isotropic (LTI) carbon material). Still another exemplary NR carrier material is material consisting of, consisting essentially of, or comprising a calcium aluminate material, an inorganic silicate, a metallic oxide, and/or a refractory hydride, sulfide, or selenide.

In aspects, a non-resorbable (“NR”) material can be a material having a Moh scale hardness of about 4-10, such as about 4-9 or about 5-9.

In another sense, NR materials can comprise a metal, certain NR ceramics (typically characterizable as bioceramics), certain NR polymers, or select inorganic materials which cannot be resorbable, or combinations of any or all thereof. For example, an inert, non-resorbable material can comprise but not be limited to an inert titanium or titanium alloy, silver, gold, iron oxide, stainless steel, bioinert oxide such as an aluminate (alumina) or zirconia ceramic material, a NR calcium phosphate such as a NR hydroxyapatite (HAP) compound, e.g. such as sintered hydroxyapatite, synthetic hydroxyapatite, or synthetic sintered hydroxyapatite with little to no bioresorbability, a bioinert carbon ceramic, silica, inert bioglass, HD-polyethylene, carbon (graphite), CFRP, or other similar or related compounds which are inert and have limited or no resorbability.

In NR carrier material LARTCMPs, the NR material can comprise greater than about 1% of the composition of the carrier material (i.e., where 1%=1 wt %). For example, according to certain embodiments, at least about 1%, greater than about 2%, at least about 5%, at least about 10%, at least about 15%, at least about 20%, at least about 25%, at least about 30%, at least about 35%, at least about 40%, greater than about 45 of the carrier is composed of a NR material, such as a NR bioceramic. In other aspects, the carrier material of a LARTCMP predominately comprises a NR material. In certain embodiments, at least about 60%, at least about 70%, at least about 75%, at least about 80%, greater than about 85% or more, as in at least about 90%, at least about 95%, at least about 99%, or about 100% (or 100%) of the composition of the carrier material can be made up of one or more non-resorbable carrier materials, such as a NR bioceramic.

A carrier can be comprised of one or more bioceramic materials, and also or alternatively a carrier material can comprise one or more bioceramic(s) composed of a single crystal bioceramic, one or more polycrystalline bioceramic(s), or a combination of the two. As noted elsewhere herein, such materials can be resorbable or non-resorbable or even include a mixture of resorbable and NR materials.

According to certain embodiments, at least about 1% of a bioceramic material used as a carrier material in the present invention comprises a single crystal material, which typically is a NR material. Such a single crystal bioceramic material can be, for example but not limited to, a sapphire or a single crystal alumina. For example, a bioceramic material used in a carrier of the present invention can comprise at least about 5%, at least about 10%, or more, for example at least about 15%, at least about 20%, at least about 25%, at least about 30%, at least about 35%, at least about 40%, or at least about 45% of the bioceramic carrier material is made up of a single crystal material. In other aspects, more than 50% of the carrier is made up of such a material (e.g., at least about 65%, at least about 75%, at least about 90%, at least about 95%, or about 100%, such as 100% of the carrier is made up of a single crystal material). According to other embodiments, the LARTCMP comprises a polycrystalline bioceramic material, which in one aspect is a NR material. Such a polycrystalline bioceramic material can be, for example but not limited to, a polycrystalline alumina. Such material can be present in any suitable amount, such as any of the amounts described in this paragraph for single crystalline bioceramic carrier materials. The carrier material also or alternatively can comprise any such amount of a combination of one or more single crystal material(s) and one or more polycrystalline material(s). In still another aspect, the carrier material also or alternatively comprises, materially comprises, predominately comprises, consists essentially of, or consists entirely of non-crystalline bioceramic material, which can be present in any of the above-recited amounts. In a further specific aspect, the non-crystalline bioceramic material is a bioglass material that is adapted to/configured to be non-resorbable. In some aspects, such materials can be configured to/adapted to be resorbable, and such materials can provide resorbable carrier materials for use in the BRIST particles described above.

### V. Bioactive Carriers

In accordance with alternative embodiments, the one or more carrier materials of the particles of the present invention can be composed of or can comprise a material that is bioactive. A “bioactive” material is any material that detectably interacts with tissues when the material is administered to the body. Typically, the interaction is a measurable tissue adherence effect. A bioactive material can be resorbable or NR. Bioactive and non-bioactive materials can also be conditionally resorbable/NR.

Bioactive materials can exhibit any suitable kind of interaction(s) with any suitable type of tissue(s). Bioactive materials can, e.g., form detectably measurable bonds with hard tissue(s) (e.g., bone), soft tissue(s) (muscles, tendons, fibrous tissues, fatty tissues, and the like), or both hard tissue(s) and soft tissue(s). Those of ordinary skill in the art will be able to recognize the differences between hard and soft tissues. In general, most tissues are classified as soft tissues with hard tissues being generally limited to most bone tissue, dentin, cementum, and enamel.

According to some embodiments, the particles can comprise, materially comprise, or, e.g., predominately comprise a bioactive material that is or comprises a bioactive glass. Such materials can comprise various combinations of SiO₂; CaO; Na₂O; and P₂O₅; often in combination with one or more of MgO; K₂O Al₂O₃; F; TiO₂; or ZrO₂. In some aspects, such a bioglass comprises a structure according to the formula SiO₂—CaO—Na₂O—P₂O₅. In some aspects, the material comprises about 25-55 wt % SiO₂, 0.1-50 wt % CaO; 2.5-25 wt % Na₂O; and about 2.5-20 wt % P₂O₅. Examples of commercially available glass materials that can be suitable are sold under the trade names Bioglass™ and Ceravital™.

According to some embodiments, the bioactive material can be a bioactive ceramic such as bioactive glasses, a hydroxyapatite, sintered beta-tricalcium phosphate (13-TCP), and glass-ceramic A-W (containing apatite (“A” and wollastonite “B”), calcium phosphate materials or similar such materials demonstrating bioactivity. As noted already, the carrier material also or alternatively can comprise a bioactive hydroxyapatite compound. Such a hydroxyapatite material can be a sintered hydroxyapatite, synthetic hydroxyapatite, or synthetic sintered hydroxyapatite.

According to certain embodiments, a bioactive carrier material, when present as an element of the particles of the present invention, can comprise greater than about 1% of the composition of the carrier material. For example, according to certain embodiments, at least about 2.5%, at least about 5%, at least about 10%, at least about 15%, at least about 20%, at least about 25%, at least about 30%, at least about 35%, at least about 40%, or at least about 45% of the carrier material is made up of a bioactive material. In other aspects, the carrier material at least predominately comprises a bioactive material. According to such embodiments, the carrier material largely consists of a bioactive material, substantially consists of a bioactive material, or consists of a bioactive material (e.g., at least about 60%, at least about 85%, at least about 100%, or 100% of the carrier material is composed of a bioactive material).

In one aspect, the bioactive carrier is a biomimetic material. Biomimetic materials are materials that exhibit tissue-like properties, such as growth. For example, some polymeric biomimetic materials undergo growth through nucleation in the body similar to the growth of bone. An example of a biomimetic material is material composed of porous polylactide-co-glycolide scaffolds that are incubated in physiological fluids.

### VI. Carrier Material Mixtures

As indicated several times above, another aspect of the invention is embodied in compositions comprising particles that have a mix of carrier materials, including mixtures of carrier materials that differ significantly in terms of composition and function. According to certain embodiments, the particles of the present invention can comprise a carrier material that is composed of a mixture of different carrier materials, such as a mixture of one or more elements selected from the group bioceramic, proteinaceous, or biodegradable polymer or copolymer; e.g., a mixture of one or more bioceramics with a proteinaceous material, a mixture of one or more proteinaceous materials with a biodegradable polymer or copolymer, a mixture of one or more biodegradable polymers or copolymers with one or more bioceramics, or a combination of any or all thereof. Such a mixture can be designed to target specific characteristics of the formulation, such as drug release characteristics, bioactivity level, or resorption time. Such particles can be validated for having such a function, such as providing a resorption time (e.g., an average resorption time) such as those specifically recited elsewhere herein (e.g., 2 years, 18 months, or 6 months).

According to further embodiments, the particles of the present invention can comprise a combination of an effective amount of a resorbable and a non-resorbable material in any ratio providing effective delivery of the drug loaded onto the carrier material as described further herein.

VII. Particles with Little or No Carrier Material

In some formulations of the present invention, the treprostinil particles of the LARTCMP pharmaceutical composition invention can comprise little- to no carrier material. According to some embodiments, the particles can comprise less than about 20% (i.e., less than about 20 wt %) of a carrier component, such as less than about 15%, less than about 10%, less than about 5%, less than about 1%, less than about 0.5% carrier material, less than about 0.1% carrier material, or even less carrier material, such as, e.g., embodiments in which the particle comprises no detectable carrier material or the particle comprises treprostinil compounds and any other APIs to be co-delivered with the treprostinil compounds with the only extraneous material that is in the particle being processing remnants and/or API degradation products. In such cases, the remainder can comprise at least in some part of one or more treprostinil compounds, and optionally further comprise one or more pharmaceutically acceptable excipients. E.g., wherein the particles comprise less than about 30% of a carrier component, the remainder of the particle composition can comprise at least about 70% of one or more treprostinil compounds or treprostinil compounds and excipients, such as, e.g., particles can comprise about 30% of a carrier and about 70% of treprostinil compound/excipient, about 25% carrier and about 75% treprostinil compound/excipient, about 20% carrier and about 80% treprostinil compound/excipient, about 15% carrier and about 85% treprostinil compound/excipient, about 10% carrier and about 90% treprostinil compound/excipient, about 5% carrier and about 95% treprostinil compound/excipient, or even less of a carrier, e.g., about 1% carrier and about 99% compound/excipient or even less carrier, such that the particles can contain no carrier material, containing only treprostinil and optionally one or more pharmaceutically acceptable adjuvants or excipients.

Any particles according to such aspects will be configured to or adapted to provide a sustained release of treprostinil. Thus, the physical properties of the particle itself, which can be attributable to design of the particle and/or the manner in which the particle is manufactured, will provide such a LARTCMP of treprostinil compound(s) even though the particle consists essentially, substantially consists of, or consists of such treprostinil compounds. One surprising aspect of this aspect of the invention is the ability to produce such low carrier material microparticles and still be able to provide a long acting release/sustained release/extended release of one or more constituent treprostinil compounds when administered to the body of a subject. The inventors have discovered that such microparticle products can be obtained by, e.g., applying microsieve microparticle production methods exemplified elsewhere herein.

### VIII. Additional APIs

As already mentioned, particles of the invention can include one or more additional non-treprostinil compound APIs in addition to the one or more treprostinil compounds in the particles. Any suitable second API can be incorporated into such a particle composition. In a first example of such an embodiment, the second API or additional APIs are compounds that can be characterized as belonging to one of the following classes of compounds: endothelin receptor antagonists (ERAs), phosphodiesterase type 5 (PDE5) inhibitors, soluble guanylate cyclase stimulators, prostacyclin receptor agonists, and prostacyclin analogs. In aspects, a second API can be selected from pitavastatin, nitric oxide, rapamycin, fasudil, beraprost, iloprost, salbutamol, curcumin, or a combination of any or all thereof. The additional API(s) also might be nucleic acid vectors or vectors comprising nucleic acids for expression of genes or for other nucleic acid functions, such as siRNA. In another aspect, the second API is a pro-angiogenic agent, such as a VEGF, a HIF protein (e.g., HIF-1a), or an FGF angiogenic protein (e.g., FGF-2).

### B. Particle Size, Shape, and Size Distribution

### I. Particle Size and Shape Characteristics, Generally

As noted above, the size and shape characteristics of the microparticles can have an influence on the properties of the composition, particularly with respect to the injectability of compositions comprising such microparticles (e.g., a microparticle dispersion). Those of skill in the art will recognize that injectability is not an absolute characteristic but one that varies in degree and that depends on the context of the formulation composition and the injection device. In general, however, particles of smaller size, e.g. less than 150 μm, will typically perform better as will compositions having more uniform size and/or shape characteristics, e.g., in the case of relatively uniform sized and sufficiently small monosphere microparticle compositions. However, as also noted elsewhere herein it also can be important that the microparticles be defined by an absolute, typical, or average minimum size, as very small sized particles can have properties that are undesirable such as negative interactions with the immune system of the subject. Thus, it can be a characteristic of compositions of the invention that they lack or are free of very small particles, e.g., particles of less than about 7 μm in average maximum dimension or diameter, less than about 6 μm in average diameter, less than about 5 μm in maximum dimension or average diameter, or less than about 2 μm in average diameter or even having a configuration such that the particles are less than about 7, 5, or 2 μm in size in any one or more dimensions.

In one aspect, LARTCMPs of the inventive composition can be characterized as being substantially uniform in size, shape, or both. The size and/or shape uniformity of the microparticles can be associated with a detectably longer or steadier release of treprostinil and/or measurably better injectability.

One way in which the importance of particle size to the present invention can be demonstrated is to characterize the limitation of the number of small particles present in the composition. This is important because, as previously discussed, particularly small particles may activate the immune system (e.g. stimulate phagocytosis by macrophages). As such, according to another aspect, the RPP particles of the composition also or alternatively can be characterized by including a limited/small number of particles (e.g., less than about 10%, less than 7.5%, less than about 5%, less than about 2%, less than about 1%, less than about 0.5%, or less than about 0.1%) that have a maximum diameter in any direction that is less than 20 μm in size. In one aspect the RPP particles of the composition can be characterized as comprising less than 5% of particles, such as less than 5%, less than 4%, less than 3%, less than 2%, or less than 1% of particles which are smaller than 10 μm in size, such as smaller than 9 μm in size, smaller than 8 μm in size, smaller than 7 μm in size, or for example smaller than 6 μm in size. In a further aspect, compositions are provided in which less than about 10%, less than 7.5%, less than about 5%, less than about 2%, less than about 1%, less than about 0.5%, or less than about 0.1% or the particles have a maximum diameter in any direction that is less than 5 μm such as less than 4 μm in size or for example less than 3 μm in size.

Monodisperse microspheres containing treprostinil for sustained drug delivery achieve a gradual release of treprostinil at a constant rate over the desired period of time. According to certain embodiments, that time is a pre-determined period of about 1 day to about 6 months. According to certain embodiments, the microsphere size and careful selection of a carrier material or carrier matrix material allow for targeted design of the release period. Microsieve emulsification technology may be used to provide high predictability and reproducibility, robustness, scalability, size control and narrow particle size distributions. Moreover, other features such as a good syringeability, limited or no phagocytosis, high encapsulation efficiency, and no drug release burst may also be achieved. According to specific embodiments, the diffusion rate of the active and the degradation rate of the polymer match the desired release period.

### II. Average Diameter or Maximum Dimension Size of Particles

One possible way to characterize compositions of the invention is the average maximum size of the particles in any one dimension or the maximum average diameter in the case of particles that are spheroid or spherical in shape. It is to be understood that these concepts can be interchanged herein such that the description of any aspect with respect to a maximum average diameter, as applied to a spheroid particle for example, is to be understood as providing corresponding support for a non-spheroid particle having an average maximum size in any one dimension of a corresponding size.

In one aspect, at least about 50%, at least about 70%, at least about 80%, at least about 90%, at least about 95%, at least about 99%, or about 100% of the particles in the composition have a maximum diameter (or average maximum dimension) of between about 5 μm and about 200 μm, such as between about 5 μm and about 150 μm, e.g., about 5 μm and about 100 μm, e.g., between about 5 μm and about 85 μm, for example between about 7 μm and about 140 μm, about 7 μm and about 105 μm, or between about 7 μm and about 70 μm, or about 8 μm and about 120 μm, such as between about 8 μm and about 90 μm. In still other common aspects the size of at least about 75%, at least about 85%, at least about 92.5%, or at least about 97.5% of the particles of the composition, or more, such as about 100% of the particles of the composition, are between about 10 μm and 150 μm, such as between about 30 μm and about 140 μm, such as between about 50 μm and about 130 μm or between about 70 μm and about 120 μm or between about 75 μm and about 110 μm.

According to certain embodiments, the present invention provides a composition comprising an effective amount of sustained release treprostinil compound monospheres, that is, the particles of the composition predominately comprise, largely consist of, substantially consist of, consist essentially of, or consist of particles that are spherical or spheroid in shape and are of a relatively consistent size, containing one or more treprostinil compounds as a depot. In some embodiments, the invention comprises a long acting release composition comprising biodegradable microparticles of treprostinil and one or more pharmaceutically acceptable excipients. Such microparticles can have a mean particle size (in terms of diameter or maximum dimension) that is less than about 150 μm, less than about 125 μm, and commonly will be less than about 100 μm, less than about 80 μm, less than about 60 μm, less than about 50 μm, less than about 45 μm, or less than about 40 μm in size (and in some cases can be less than about 35 μm or less than about 30 μm in size). As noted elsewhere, particles of the invention also will typically have a minimum size in most cases, e.g., at least about 80%, at least about 85%, at least about 90%, at least about 95%, at least about 99%, or about 100% of the microparticles will have an average diameter, maximum dimension, or minimum dimension of at least about 5 μm, such as at least about 8 μm, at least about 9 μm, or at least about 10 μm (and in some cases at least about 12 μm, at least about 14 μm, or at least about 15 μm). Any of these minimum and maximum sizes/dimensions can be combined to provide ranges of particle sizes that will reflect different aspects/characteristics of the inventive compositions.

According to certain specific exemplary embodiments, the average microsphere diameter is in the range of between about 10 μm-about 200 μm, for example the average maximum diameter of the particles is about 10 μm, about 20 μm, about 30 μm, about 40 μm, about 50 μm, or about 60 μm, for example about 65 μm, about 70 μm, about 80 μm, about 90 μm, or about 100 μm, as in about 110 μm, about 120 μm, about 130 μm, about 140 μm, or about 150 μm, such as for example approximately 160 μm, approximately 170 μm, approximately 180 μm, approximately 190 μm, or approximately 200 μm. In some embodiments, the average microsphere diameter is in the range of between about 75 μm to about 105 μm, such as the average microsphere diameter being about 75 μm, about 76 μm, about 77 μm, about 78 μm, or about 79 μm, as in about 80 μm, about 81 μm, about 82 μm, about 83 μm, about 84 μm, about 85 μm, about 86 μm, about 87 μm, about 88 μm, about 89 μm, about 90 μm, about 91 μm, about 92 μm, about 93 μm, about 94 μm, about 95 μm, about 96 μm, about 97 μm, about 98 μm, about 99 μm, about 100 μm, about 101 μm, about 102 μm, about 103 μm, about 104 μm or about 105 μm.

According to a particular embodiment, at least about 80% of the particles in the composition are between 5 μm and 200 μm in size, for example at least about 80%, at least about 82%, at least about 84%, at least about 86%, at least about 88%, at least about 90%, at least about 92%, at least about 94%, at least about 96%, at least about 98%, or at least about 99% of the particles in the composition are between about 5 μm and about 200 μm in size. According to further embodiments, at least about 90%, for example about 91%, about 92%, about 93%, about 94%, about 95%, about 96%, about 97%, about 98%, about 99%, or about 99.9% of particles are between about 5 μm and about 200 μm in size. According to yet further embodiments. At least about 65%, for example at least about 65%, at least about 70%, at least about 75%, at least about 80%, at least about 85%, at least about 90%, at least about 95%, at least about 97.5%, or at least about 99%, for example at least approximately 99.5% of the particles of the composition have an average diameter in the range of about 20 μm to 150 μm. In further embodiments, at least about 70%, for example at least about 70%, at least about 75%, at least about 80%, at least about 85%, at least about 90%, at least about 95%, at least about 97.5%, at least about 99%, or at least about 99.5% of the particles in the composition have an average diameter in the range of between about 20 μm to about 150 μm. According to still further embodiments, at least about 70% of the microparticles (e.g., the monospheres) have a maximum diameter of between about 50 μm and about 130 μm in size. Clearly ranges of acceptable particles can be generated from such disclosure. For example, in one aspect the particles have an average diameter in the range of about 20 μm-150 μm or in the range of about 70-110 μm.

According to certain embodiments, the maximum average dimension of three agglomerated particles of the composition(s) described herein is less than about 500 μm, for example less than about 490 μm, less than about 480 μm, less than about 470 μm, less than about 460 μm, or less than about 450 μm, for example less than about 440 μm, less than about 430 μm, less than about 420 μm, less than about 410 μm, or even less, such as less than about 400 μm in diameter. For example, the maximum average dimension of three agglomerated particles of the composition(s) described herein is less than about 400 μm, such as less than about 390 μm, less than about 380 μm, less than about 370 μm, less than about 360 μm, less than about 350 μm, less than about 340 μm, less than about 330 μm, less than about 320 μm, less than about 310 μm, or even less than about 300 μm. This size dimension is directly related to the maximum size dimension of the particles of the composition (e.g., spherical particles having a diameter of about 100 μm, would expect to have a maximum three-particle aggregation size of about 300 μm). The aggregation size of multiple particles is relevant in the sense that if particles in a microparticle composition, such as an aqueous liquid dispersion, aggregate, the possible size of aggregates can determine whether the inner diameter of any part of an injection delivery system (e.g., a syringe) will become clogged. In other words, the size of potential aggregates (as well as the frequency of aggregation) is what typically determines injectability.

### III. Size Distribution of Particles and Methods of Production

Compositions of the invention also or alternatively can be characterized on the basis of the size distribution of particles in the composition. In this respect it is worth noting that many compositions of the invention will comprise particles that vary in size due to differences that arise in the manufacturing process, handling, or for similar reasons. As noted already, compositions with relatively uniform sizes can offer advantageous properties including in aiding in injectability of the composition. Many microparticle products have a very large distribution of sizes (e.g., at least about 10% of the particles in such a composition can vary in average diameter or average maximum size by about 5×, about 7×, about 10×, about 12×, about 15×, or even about 20×). While such compositions are not necessarily excluded from the scope of this disclosure, an aspect of the invention is the provision of compositions of biocompatible LARTCMPs having relatively uniform size distributions.

The relative uniformity in particle size of the LARTCMPs of the present invention can be characterized by describing the coefficient of variation in particle size in the composition. E.g., in aspects, the particle size coefficient of variation (CV) of microspheres of the composition is less than about 50%, e.g., less than about 45%, less than about 40%, less than about 35%, less than 30%, less than about 25%, less than about 20%, less than 15%, less than about 12.5%, less than about 10%, or less than about 7.5%, e.g., less than about 7%, less than about 6%, or less than about 5%.

In aspects, the invention provides compositions wherein the coefficient of variation (CV) in the average diameter of the particles of the composition (or average maximum size of the particles in any dimension) is less than about 20%. In more particular aspects the invention provides compositions comprising spheroid biocompatible LARTCMPs wherein the CV in the average diameter of the particles is less than about 15%, e.g., less than about 12%, less than about 10%, less than about 7.5%, or even less than about 5%.

Provision of a uniform distribution of particles can be achieved by any means. Examples of suitable methods are described below, but it can be possible that alternative methods will be developed in the future or currently exist that will provide particle compositions with the same or comparable properties.

One such production method comprises flow focusing devices in which a fluid is injected through a nozzle into a stream of another fluid, and droplets are detached by Rayleigh instability. While essentially perfectly monodisperse particles can be obtained in the laboratory by employing such a production method, scaling up such a method for industrial purposes is extremely difficult, mainly due to the low production rate and the need for exact control over two different fluid streams (crucial to obtain a certain droplet size). At present, numerous complex devices operating in parallel, each with the exact same supply of two different fluid streams, are required in order to produce volumes suitable for pharmaceutical applications. Accordingly, the current application of such a method can be limited.

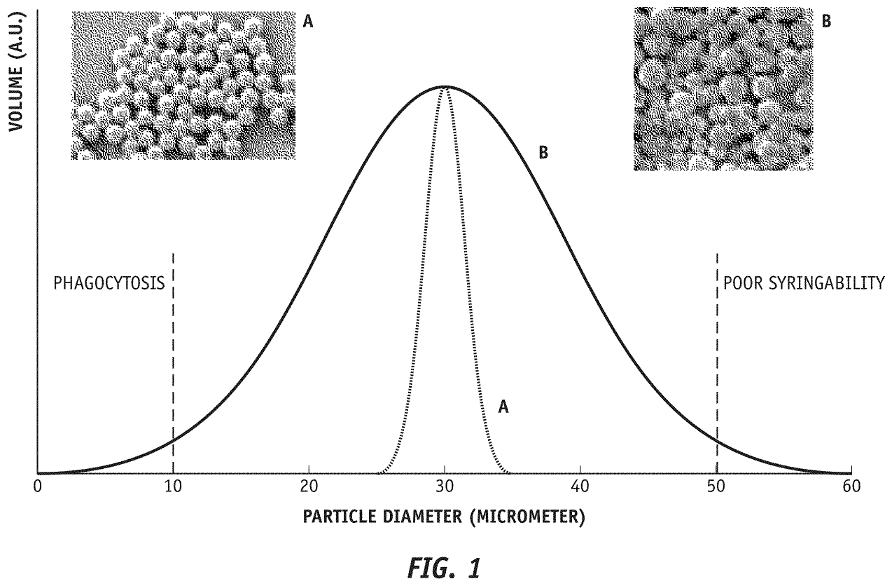

In an alternative aspect, the particles are produced by a method that comprises a micro-sieve (or microsieve) emulsification method. The inventors have discovered that micro-sieve emulsification methods, which are described further elsewhere herein and in the art, can result in LARTCMPs that exhibit a relatively narrow particle size and shape distribution as compared to other methods, such as solvent extraction/evaporation and high-speed homogenization micronization methods. A comparison of the sizes and size distribution of similar content microparticles produced by both methods is shown in FIG. 1, with graph (A) illustrating microparticle size distribution obtained using Nanomi's Microsieve™ emulsification method used in the production of particles of the invention (CV of about 5%) and graph (B) illustrating microparticle size distribution obtained using conventional emulsification with homogenizers (CV of about 30%). FIG. 1 also indicates via dashed lines size limits for phagocytosis (about 10 μm) and poor syringability for depot formulations (about 50 μm). In aspects, the narrow size distribution of particles manufactured using a microsieve emulsification method provide particles with minimal risk of phagocytosis, the ability to be dispensed using a syringe as described elsewhere herein with minimal syringe clogging.

It is expected that the size distribution of LARTCMPs produced by such methods will exhibit a similar pattern of relative uniformity in terms of size and shape. Compositions comprising microparticles produced by microsieve production methods represent another aspect of the invention.

Another approach for obtaining uniform and monodisperse droplets and particles is offered by membrane emulsification. In membrane emulsification, a fluid is forced through a porous membrane. The droplets emerging on the other side of the membrane surface are wiped off by the shear forces induced by a stream of another fluid across the membrane. Typical membranes used are similar to those used for filtration purposes. Control over droplet size and uniformity is better than for high-shear homogenizers but inferior to the control that can be achieved by single microfluidic devices.

Microsieve technology overcomes limitations in membrane emulsification and fluid injection micronization methods. This is a relatively new technology, but since it is known and described in the art it is only briefly described herein for the convenience of readers of this disclosure and to evidence the enablement of aspects of the invention.

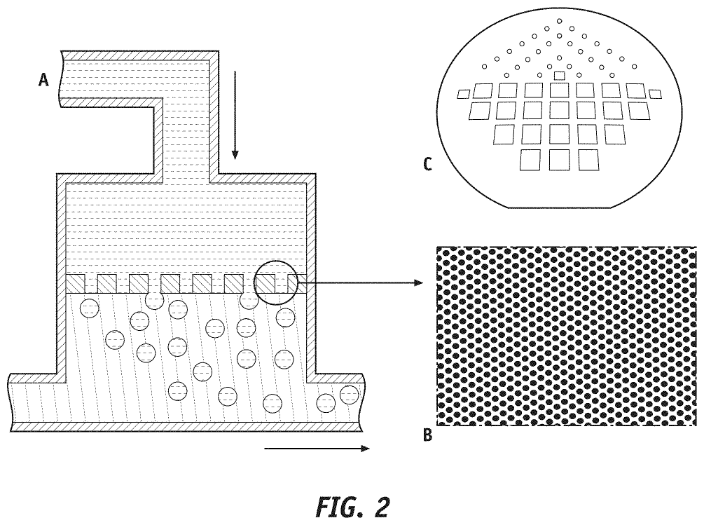

Generally, in microsieve emulsification monodisperse droplets are generated by dispersing one fluid into a second, immiscible fluid through a precise microsieve (see FIG. 2). Microsieves are membranes, typically silicon-based membranes, which are fabricated by proven precise semiconductor technology in a cleanroom environment, typically by means of photolithographic techniques. By use of such membranes in the particle composition production process, excellent uniformity of pore size and shape is obtained in a highly reproducible manner. The schematic of FIG. 2 illustrates Nanomi's Microsieve™ emulsification process (A), where a fluid is emulsified through a silicone Microsieve™ membrane with uniform pores (B). Section (C) of the schematic illustrates a wafer containing Microsieves™ fabricated by semiconductor technology.

Because every pore in this process is typically the same every droplet generated by the membrane is also the same (or at least substantially the same), resulting in highly uniform, reproducible, and size-controlled droplets or, after an appropriate solidification step, particles. The method works with various droplet sizes, such that the resulting particle size is driven primarily if not exclusively by the membrane design. The process also does not typically require a cross-flow in order to produce particles (which is a feature of other competing methods).

Applying this method to the production of LARTCMPs can be performed through preparing a treprostinil compound solution in which the treprostinil compound(s) and optionally other particle components/ingredients are dissolved in a suitable volatile solvent and then emulsified by contact with an aqueous solution comprising suitable surfactant(s). The solvent is eliminated from this composition, typically through evaporation, resulting in the formation of solid microparticles.

Methods relating to the production of microparticles by microsieve emulsion technology are provided in International Patent Application WO 2018/193389, which is incorporated by reference herein with respect to the production of LARTCMPs. The inventors have discovered that the methods of the '389 application are useful in the generation of such microparticles. As such, the application of the '389 application methods to such compositions and compositions comprising LARTCMPs produced by the '389 application method represent additional aspects of this invention.

Alternative methods for producing microparticles that can meet some of the features of the inventive aspects provided herein are known in the art and touched on elsewhere herein (including in the Background of this disclosure). One source of disclosure of such alternative prior art methods is described in WO 2005/115599. In one embodiment, the micro- and/or nanospheres of the present invention are prepared using the methods disclosed in the U.S. Pat. No. 8,100,348 which also is incorporated herein in its entirety with respect to production methods and microparticle features.

Particles of the compositions described herein can have a size distribution such that at least about 20%, such as at least about 22%, at least about 24%, at least about 26%, at least about 28%, at least about 30%, at least about 32%, at least about 34%, at least about 36%, at least about 38%, at least about 40%, at least about 42%, at least about 44%, at least about 46% at least about 48% or at least about 50%, such as at least about 52%, at least about 54%, at least about 56%, at least about 58%, at least about 60%, at least about 62%, at least about 64%, at least about 66%, at least about 68%, at least about 70%, at least about 72%, at least about 74%, at least about 76%, at least about 78%, or at least about 80% of the particles of the composition have a maximum particle diameter that is within about 50%, for example within about 50%, within about 48%, within about 46%, within about 44%, within about 42%, within about 40%, within about 38%, within about 36%, within about 34%, within about 32%, within about 30%, or within approximately 28%, within approximately 26%, within approximately 24%, within approximately 22%, within approximately 20%, within approximately 18%, within approximately 16%, within approximately 14%, within approximately 12%, or within approximately 10% of the average particle diameter of the particles in the composition. According to specific embodiments, at least about 65% of the particles of the composition have a maximum particle diameter that is within 35% of the average particle diameter of the particles in the composition. According to alternative specific embodiments, at least about 70% of the particles of the composition have a maximum particle diameter that is within about 33% of the average particle diameter of the particles in the composition. According to yet further embodiments, at least 33% of the particles have a maximum diameter that is within about 15% of the average particle diameter of particles in the composition. In yet another embodiment, at least about 40% of the particles have a maximum diameter that is within about 20% of the average particle diameter of the particles in the composition.

In some aspects, the long acting release treprostinil microparticles also or alternatively can have a size distribution with a coefficient of variation (with respect to the average diameter of the particles or maximum dimension of the particles) of less than about 5%, for example less than about 5%, less than about 4.9%, less than about 4.8%, less than about 4.7%, less than about 4.6%, less than about 4.5%, less than about 4.4%, less than about 4.3%, less than about 4.2%, less than about 4.1%, or less than about 4.0%, such as for example less than about 3.9%, less than about 3.8%, less than about 3.7%, about 3.6%, less than about 3.5%, less than about 3.4%, less than about 3.3%, less than about 3.2%, less than about 3.1%, or less than about 3.0%, as in less than about 2.9%, less than about 2.8%, less than about 2.7%, less than about 2.6%, less than about 2.5%, less than about 2.4%, less than about 2.3%, less than about 2.2%, less than about 2.1%, or less than about 2.0%, for example less than about 1.9%, less than about 1.8%, less than about 1.7%, less than about 1.6%, less than about 1.5%, less than about 1.4%, less than about 1.3%, less than about 1.2%, less than about 1.1%, or less than about 1.0%, such as for example less than about 0.9%, less than about 0.8%, less than about 0.7%, less than about 0.6%, less than about 0.5%, less than about 0.4%, less than about 0.3%, less than about 0.2%, less than about 0.1%, or a size distribution with a coefficient of variation of less than about 0.05%.

According to certain embodiments, less than about 20% of particles, such as less than about 15%, less than about 10%, or for example less than about 5% of the particles of the present invention have an average maximum dimension/diameter that is below 50 μm. According to certain embodiments, less than about 20%, less than about 15%, less than about 10%, less than about 5%, less than about 2.5%, or less than about 1% of the particles in the composition have an average maximum dimension/diameter that is less than about 60 μm, less than about 65 μm, or less than about 70 μm. In some embodiments, most, substantially all, essentially all of the particles have a minimum diameter of at least about 70 μm, at least about 75 μm, or at least about 80 μm.

According to certain embodiments, less than about 5% of the particles of the present invention have an average maximum diameter that is below 5 μm, a point at which phagocytosis risk is expected to be increased. For example, less than about 5%, less than about 4%, less than about 3%, less than about 2%, less than about 1%, or less than about 0.5% of the particles of the present invention have an average maximum diameter below 5 μm. According to certain embodiments, less than about 5% of the particles have a maximum diameter that is greater than 150 μm. For example, less than about 5%, less than about 4%, less than about 3%, less than about 2%, less than about 1%, or less than about 0.5% of the particles of the present invention have an average maximum diameter greater than 150 μm. According to further embodiments, less than about 10% of the particles have a size that is greater than 140 μm, for example less than about 10%, less than about 9.5%, less than about 9%, less than about 8.5%, less than about 8%, less than about 7.5%, less than about 7%, less than about 6.5%, less than about 6%, less than about 5.5%, less than about 5%, less than about 4.5%, less than about 4%, less than about 3.5%, less than about 3%, less than about 2.5% or even less, such as less than approximately 2%, less than approximately 1.5%, or less than approximately 1% of particles have a maximum diameter greater than 140 μm. According to a specific embodiment, less than 7.5% of the particles have a size that is greater than 140 μm. According to a specific embodiment, less than 20% of the particles have a size that is less than 50 μm and greater than 140 μm.

In some aspects, the particles of the inventive compositions can alternatively be described by their size relative to one another. According to certain embodiments, the microparticles of the compositions/formulations described herein are monodisperse, having at least about 85%, at least about 90%, at least about 95%, or more (e.g., at least about 97.5% or at least about 99%) of the RPP particles having an average maximum diameter that is within about 15 μm, within about 12.5 μm, within about 10 μm, or within about 5 μm of one another.

### IV. Shape of Particles/Distribution of Particle Diameters

In addition to the possible size characteristics and size distribution characteristics described above in certain aspects the particles of the inventive compositions provided herein can also or alternatively be characterizable based on the shape of some, most, largely all, substantially all, or all of the particles in the composition.

Particles of the invention can have any suitable shape. For example, the particles can have a “pollen” shape, a squircle shape, a disc shape, or other shape. In a typical aspect the particles have a spherical or spheroid shape. The uniformity of shape of particles can be determined by comparison of the dimensions of the particles. In one aspect, the composition materially comprises, predominately comprises, largely consists of, substantially consists of, consists essentially of, or consists of particles having a relatively similar proportion in most dimensions, in at least 65% of dimensions, at least 75% of dimensions, at least 90% of dimensions, or in all dimensions. Dimensions in this respect means dimensions in all planes of the particles' three-dimensional shapes. E.g., at least about 50%, at least about 70%, at least about 85%, at least about 95%, or at least about 100% of the particles in aspects can be characterizable as having the same shape.

Commonly in addition to having the same shape the particles will also have the same size, such that the proportions described above can be 1:1 or about 1:1. For example, where most, largely all, nearly all, or all of the particles in the composition are spherical/spheroid less than about 10% of particles can typically have a maximum diameter that is more than 40% greater than the average diameter of particles in the composition or less than about 40% of the average diameter of particles in the composition. For example, less than about 10%, less than about 9%, less than about 8%, less than about 7%, less than about 6%, less than about 5%, less than about 4%, less than about 3%, less than about 2%, or less than about 1%, such as less than about 0.9%, less than about 0.8%, less than about 0.7%, less than about 0.6%, less than about 0.5%, less than about 0.4%, less than about 0.3%, less than about 0.2%, less than about 0.1%, less than about 0.05%, or less than about 0.01% of particles have a maximum diameter that is more than about 40%, such as more about 42%, more than about 44%, more than about 46%, more than about 48%, more than about 50%, more than about 52%, more than about 54%, more than about 56%, more than about 58%, or more than about 60%, for example more than about 62%, more than about 64%, more than about 66%, more than about 68%, more than about 70%, more than about 72%, more than about 74%, more than about 76%, more than about 78%, more than about 80%, more than about 82%, more than about 84%, more than about 86%, more than about 88%, or more than about 90%, for example more than approximately 92%, more than approximately 94%, more than approximately 96%, more than approximately 98%, more than approximately 99% larger or smaller than the average diameter of particles in the composition. According to one embodiment, less than 1% of particles have a maximum diameter that is more than 66% greater than the average diameter of particles in the composition or less than about 66% of the average diameter of particles in the composition. According to one embodiment, less than about 10% of the particles have a maximum diameter more than 50% greater or less than about 50% less than the average diameter of particles in the composition.

As previously illustrated in figures and alluded to by description, the particles of the present invention can have a substantially round or spherical shape. Determination of particle shape can be achieved through any suitable method, including simple visual microscopic inspection. Compositions having substantially uniform spherical/spheroid monosphere particles of substantially similar sizes can have measurably improved properties, e.g., more uniform drug release or reduced agglomeration.

In one exemplary aspect, the invention provides compositions in which the diameter of at least about 80% of the particles of the composition can vary by no more than about 5% in any direction. That is, for example, at least about 80%, for example at least about 82%, at least about 84%, at least about 86%, at least about 88%, or for example at least approximately 90%, at least approximately 92%, at least approximately 94%, at least approximately 94%, at least approximately 96%, at least approximately 98%, at least approximately 99% or at least approximately 99.5% of the particles of the present invention vary by no more than about 15%, for example vary by no more than about 14%, about 13%, about 12%, about 11%, about 10%, about 9%, about 8%, about 7%, about 6%, about 5%, about 4%, or even less, for example about 3%, about 2% or vary by no more than 1% in any direction, conferring a primarily spherical shape to the particles. According to specific embodiments, at least about 80% of the particles of the composition vary by no more than 15% in any direction. According to more specific embodiments, at least about 90% of the particles of the composition vary by no more than about 5% in any direction. According to one embodiment, at least about 80% of the particles of the composition vary by no more than about 2% in any direction, and according to yet further specific embodiments, at least about 85% of the particles of the present invention vary by no more than about 1% in any direction.

In another aspect, the invention provides particles wherein the diameter (or average maximum dimension, average dimension, and/or average minimum dimension) of at least 80% of the particles of the composition (e.g., at least about 90% of the particles or at least about 95% of the particles) varies by no more than 15% in any direction. In more particular aspects at least about 85%, at least about 85%, or at least about 99% of the particles have diameters that vary by 5% or less with respect to the average diameter of the particles in the composition. In still more precise aspects, at least about 50%, at least about 60%, at least about 70%, at least about 85%, or at least about 95% of the particles can have diameters that are within about 2% of the average particle diameter or even within about 1% of the average particle diameter.

### C. Controlled Release

An important aspect of the characteristics of the microparticles of the present invention is that the controlled release of treprostinil contained therein. Such controlled release (also referred to as a depot formulation), provides for numerous benefits of the present invention over existing art in that it alleviates the requirement of more constant or continuous treatment administration to overcome the short half-life of treprostinil.

The pharmaceutical composition comprising LARTCMPs wherein treprostinil is released slowly and in a controlled manner over the course of time, can be described as having a “controlled release”. According to certain embodiments, term “controlled release” means the release of treprostinil at a rate slower than immediate release, such as a release of treprostinil after a single dose over the course of about 24 hours, about one week, about two months, about three months, about four months, about five months, or even up to about six months.

According to further aspects, loaded treprostinil can be released from microparticles and/or nanoparticles upon administration to the patient, that is, upon exposure to the physiological environment of the patient's body. In certain aspects, the release of treprostinil from the micro- or nanoparticle carriers is a controlled release, such controlled release transpiring over the course time periods such as 1 day to about 6 months (e.g., about 1 day to about 90 days, about 1 day to about 120 days, or about 1 day to about 300 days), providing the patient with a relatively continuous dose of treprostinil (e.g., a dose that varies by no more than about 20%, no more than about 15%, no more than about 10%, no more than about 5%, no more than about 2%, or even no more than about 1% day-to-day during the applicable treatment period).

The long acting release characteristic of the pharmaceutical composition(s) and formulation(s) of the present invention may support a therapeutic administration schedule of such composition(s) and formulation(s) wherein the composition/formulation is administered no more than once daily, for example about once daily, about once every 48 hours, about once every 3 days, about once every 4 days, about once every 5 days, about once every 6 days, about once per week, about once every 1.5 weeks, about once twice per month, about once per month, about once every 1.5 months, about once every 2 months, about once every 2.5 months, about once every 12 weeks, about once every 3.5 months, about once every 4 months, about once every 18 weeks, about once every 5 months, about once every 21 weeks, about once every 22 weeks, about once every 23 weeks, or about once every 6 months.

The long acting release characteristics of the present invention are such that treprostinil can be released slowly and in a controlled manner over the course of time after being administered to a patient, for example a single dose administered can slowly release treprostinil over the course of about 24 hours, about 48 hours, about 3 days, about 4 days, about 5 days, about 6 days, about one week, about 1.5 weeks, about one month, about 1.5 months, about 2 months, about 2.5 months, about 12 weeks, about 3.5 months, about 4 months, about 18 weeks, about 5 months, about 21 weeks, about 22 weeks, about 23 weeks, or about 6 months.

### 5. Formulations, Drug/Device Combinations, and Kits

The LARTCMPs can be presented as or incorporated into a variety of different formulations that represent additional facets of the invention. In one aspect, the invention provides a composition that largely consists of, substantially consists of, consists essentially of, or consists entirely of LARTCMPs. In one embodiment, such a composition is presented as a dry formulation.

Another aspect of the invention is embodied in compositions that comprise a formulation of one or more LARTCMPs. A “formulation” or “formulation composition” in the context of this disclosure is a composition that comprises (1) an effective amount of LARTCMPs (e.g., BRIST particles that comprise or do not comprise a carrier material or BNIST particles); (2) a “vehicle” component, which can be a gel, but which typically is a “diluent” component, which can include one or more liquid solvents for forming a liquid, and typically aqueous, suspension/dispersion of the LARTCMPs; and optionally (3) one or more excipients. Formulations of the invention can include any suitable vehicle component(s), such as any suitable diluent components, and can optionally comprise one or more excipients. In one aspect, the invention provides formulation compositions that are suitable for injection, such as injection through a needle.

For example, LARTCMPs can be formulated with a pharmaceutically acceptable diluent, such as water for injection, and optionally one or more excipients, in order to form a liquid dispersion (sometimes called a liquid solution). A liquid dispersion can be placed into a delivery device, such as a needle. Alternatively, a liquid dispersion composition or separate components for preparing such a composition (e.g., separate containers containing dry microparticles and a diluent composition) can be provided with a delivery system as a “kit”, a composition/device combination, or a “system.” Each of these types of compositions will be further exemplified below.

Examples of typical diluents for injectable compositions include water for injection. In other aspects, diluents can also or alternatively include one or more oils, such as petroleum, animal, vegetable, plant, or oils of synthetic origin.

According to certain embodiments compositions and formulations of the present invention are suitable for injectable use (e.g., intravenous, intra-arterial, intramuscular, etc. as exemplified elsewhere herein) such that they comprise sterile aqueous solutions or suspensions and sterile powders or dried material for the immediate or extemporaneous preparation of sterile injectable solutions or suspensions. According to primary embodiments, the compositions and/or formulations of invention are sterile so as to be appropriate for injection into a living being. According to certain embodiments, the compositions/formulations of the present invention are fluid to the extent that easy syringeability (ease of dispensing via a syringe system comprising a needle) exists; in alternative embodiments, the compositions/formulations of the present invention are not liquid; however according to either form, according to most common embodiments the material in either form is suitable for injection into a living being who can benefit from receiving such compositions/formulations.

To maintain suitability for administration to a living being who can benefit from receiving the compositions/formulations described herein, according to certain embodiments, compositions of the present invention are stable under the conditions of manufacture and storage and comprise elements which support protection against the contaminating action of microorganisms, such as bacteria and fungi, which may include excipients acting as preservatives as well as packaging means to prevent and control contamination. Such stability is maintained through, for example according to certain aspects, effectively sealing the components of the compositions/formulations in effective packaging. Also or alternatively, such suitability may be provided, maintained, or supported through the use of appropriate excipients, e.g. anti-microbials, which aid in the prevention of contamination.

### I. Microparticle Compositions (MPCs)

As noted, microparticles having the characteristics described above may be referred to as a long acting release treprostinil compound microparticles or LARTCMPs. In some aspects of the invention, LARTCMPs are provided as a dried particle composition (e.g., in a freeze-dried or cryodessicated form or otherwise dried form). According to some embodiments, one or more excipients can be present along with the LARTCMPs in dried form, either separate from or as part of the LARTCMP(s). Such LARTCMP-only or LARTCMP-plus-excipient combinations may be referred to as a microparticle composition or “MPC.” The term “MPC” is used to avoid confusion with compositions in which LARTCMPs are dispersed or suspended in a liquid diluent/solvent, gel, or other suitable formulation vehicle. MPC compositions are useful for storage and shipment of LARTCMPs. They also can be used directly as implants or in certain injection systems. Furthermore, MPCs can be components of compositions, such as kits, that comprise the MPC and other components, such as, e.g., a needle deliver system, a storage container, and possibly one or more vehicles for forming a dispersion, gel, or other type of suitable formulation for administration of the LARTCMP(s) to a subject. As such, MPCs are an important aspect of the invention.

Compositions of microparticles described above can be in any suitable form or can assume a variety of forms depending upon how such dry compositions themselves are formulated. In one aspect, the composition of microparticles is a substantially dry or dry composition (e.g., a composition having a water or liquid content of less than about 1%, less than about 0.1%, less than about 0.001%, or less than about 0.0001% or having other indicators that would reflect that the composition is dry). Dried particle compositions can be prepared by any suitable method, including dry production or by application of drying techniques known in the art.

### II. Liquid Formulations

According to one aspect of the invention, MPCs are formulated as a liquid dispersion. These formulations (note here the term “formulation” applies as the composition includes MPCs, a vehicle, in this context typically a diluent, and optionally one or more excipients) include dried particle compositions and compositions in which the dried particles are dispersed or suspended in a vehicle or diluent. The term “reconstitute” is used herein to describe the formulation of such a liquid dispersion, which is typically an aqueous dispersion. While the term “dispersion” is used herein, it should be recognized that formulation compositions also can be suspensions, as a vehicle can be a gel or other vehicle that is an alternative to a liquid solvent.

The microparticle compositions described above can be incorporated into a variety of different formulations that represent additional facets of the invention. The vehicle and thus the form of the formulation can be any suitable form and formulation for use of the microparticles (e.g., for the treatment of a treprostinil compound-treatable condition). As simple examples, the formulation can be an aqueous formulation, an emulsion, or any formulation which supports the administration of the LARTCMP(s) in a form suitable for administration to a target in need of such treatment.

Liquid dispersion/solution formulations can be formed by any suitable diluent or combination of diluents combined with a composition of particles of the invention. As noted, in some aspects, the liquid dispersion is an aqueous liquid composition, with the primary diluent being water, typically a purified water, such as a water that is classified as “water for injection.” Other formulations can include any other suitable sterile or otherwise pharmaceutically acceptable vehicles. Additional diluents (sometimes referred to as carriers or vehicles in the art) can include but are not limited to water, saline, sugar solutions such as aqueous dextrose and related sugars, and glycols such as propylene glycol or polyethylene glycol.

According to certain embodiments, the formulation comprises a dispersion or suspension further comprising excipients such as one or more emulsion-forming materials or emulsion phase components. In one aspect, a liquid composition can comprise a suitable oil component, which can be a vegetable oil, another plant oil, or an oil of synthetic origin, for example peanut, soybean or mineral oil.

In still other exemplary aspects, the formulation can include one or more ion exchange resins or sparingly soluble derivatives (e.g., as a material that would in the art be characterized as a sparingly soluble salt).

Vehicles (e.g., diluents), carriers, and excipients are typically materials that exhibit pharmaceutically acceptable levels of biocompatibility, biodegradability, mechanical properties, cosmetic appearance, and interface properties, if applicable/relevant, as will be within the ability of those of skill in the art to determine.

As stated previously, a liquid dispersion or suspension composition/formulation can be presented in a ready-to-use form, preloaded into a delivery device, such as a syringe equipped with a needle. Alternatively, separate components for preparing such a composition (e.g., separate containers containing dry microparticles and a diluent composition) can be provided with a delivery system as a “kit”, a composition/device combination, or a “system.” Each of these types of presentations are exemplified further below.

A liquid microparticle dispersion/suspension formulation can comprise any suitable amount of diluent(s) and any suitable amount of microparticles (and thus any suitable concentration of microparticles). According to embodiments, about 0.5 mL to approximately 10 mL of liquid can be used to, e.g., reconstitute a dry MPC composition. For example, about 0.5 mL, about 1 mL, about 1.5 mL, about 2 mL, about 2.5 mL, or about 3 mL of liquid, such as about 4 mL, about 4.5 mL, about 5 mL, about 5.5 mL, about 6 mL, about 6.5 mL, about 7 mL or about 7.5 mL, as in for example about 8 mL, about 8.5 mL, about 9 mL, or about 9.5 mL or approximately 10 mL of liquid can be used for reconstitution/dispersion of microparticle composition material.

A liquid dispersion/suspension of the invention can have any suitable suspension density. According to embodiments, the suspension density can range from about 10 wt % to about 50 wt %, e.g., approximately 5 wt %, approximately 7.5 wt %, approximately 10 wt %, approximately 12.5 wt %, approximately 15 wt %, approximately 17.5 wt %, approximately 20 wt %, approximately 22.5 wt %, or approximately 25 wt % final suspension density, that is, about 27.5 wt %, about 30 wt %, about 32.5 wt %, about 35 wt %, about 37.5 wt %, about 40 wt %, about 42.5 wt %, about 45 wt %, or about 47.5 wt % suspension density, such as about 50% suspension density or about 52.5 wt % or about 55% wt % suspension density.

According to further embodiments, liquid formulations of LARTCMPs as described are suitable for injectable administration to a subject, such as a human patient. In further embodiments, the LARTCMPs are adapted to (formulated to) be administered by subcutaneous or intramuscular route. The liquid formulations described herein for administration to a living being is, according to primary embodiments, are in a form suitable for such administration, that is in an effectively stable form free of contamination as previously described.

As described previously, upon constitution a formulation composition can become, in some embodiments, an aqueous suspension. In alternative embodiments, when an oil or lipid-based diluent is utilized, the formulation becomes an emulsion. The formulation upon reconstitution may or may not, according to some embodiments, become a gel. Many formulation compositions of the invention can be suitable for administration via a parenteral route, such as subdermal or intramuscular injection, and can include other excipients or vehicles suitable in the context of formulations for such routes of administration.

### III. Optionally Excluded Features or Elements

The LARTCMPs of the present invention provide formulation flexibility to support a plurality of administration means. Such flexibility provides a means of targeting a wider population of targets who can benefit from treprostinil delivery. In some target conditions, there can be means of administration better suited than others, for example those with particularly restricted breathing who may benefit from treprostinil treatment may not benefit from a formulation designed to be administered via inhalation.

It is therefore the case that according to certain embodiments, a formulation can be characterized as not being an aerosolized formulation and/or can be characterized as lacking the attributes and/or components of an aerosol formulation (e.g., aerosol formulation components). According to certain aspects, the compositions comprised therein would not be suitable for administration via inhalation.

In aspects, a formulation composition of the invention also or alternatively can be described as not being delivered by infusion or suitably formulated for delivery by infusion. In certain embodiments, the formulation composition of the invention is also or alternatively not adapted to be inhaled or characterized as being administered via inhalation. In still another aspect, the formulation composition can also or alternatively be characterized as not being suitable for (e.g., adapted for, formulated for, or configured for) intravenous administration.

Formulations of the invention can be free of certain materials. In one aspect, formulation compositions of the invention are characterized as being free of, e.g., a hydrogel. In another aspect, the formulation composition is also or alternatively characterized as being free of a polymer gel comprising crosslinked polymer chains, such gels being observed as elements of carrier materials in other drug-loaded microparticle approaches. According to certain embodiments, the formulation(s) do not comprise a collagen hydrogel, a calcium phosphate hydrogel, a poly(2-hydroxyethyl methacrylate, a poly(2-hydroylpropyl methacrylate, a polyacrylic acid, a PEC or urethane polymer, a carbomer (e.g. carbomer 940 or carbomer 980), a gelatin, an alginate, a hydroxyethylcellulose, or a carboxymethylcellulose.

According to certain embodiments, the LARTCMP particles are free of any hydrogel component or such other materials described in this paragraph.

The LARTCMPs described herein according to some embodiments are not formulated to be administered in the form of an oral tablet, or other oral delivery form.

### IV. Administration: Needles/Injection Devices or Other Delivery Systems

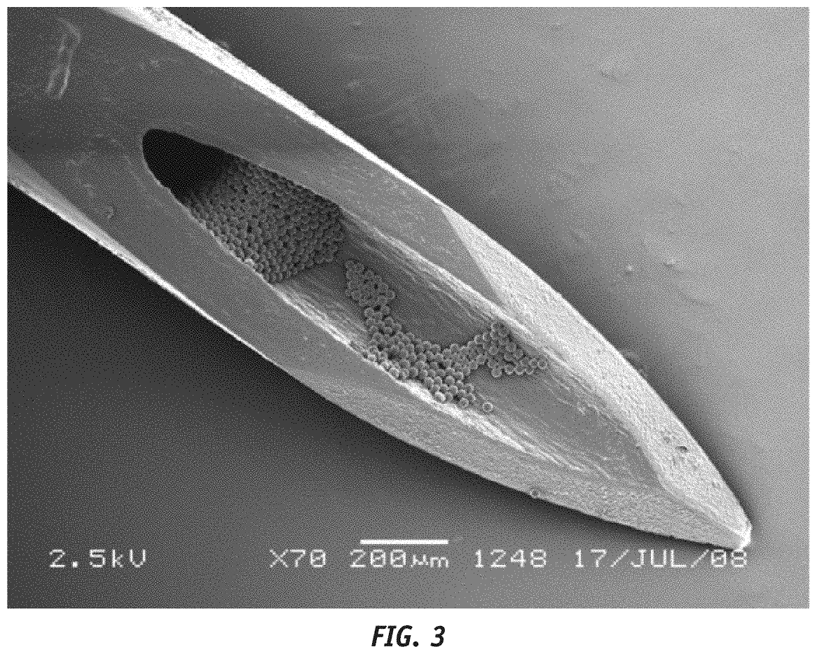

As discussed previously, syringeability, or the ability to administer the composition(s) described herein via syringe without blockage due to agglomeration is, in aspects, a feature of compositions of this invention (as also may be true of the more general concept of injectability also discussed herein). As compositions/formulations of the LARTCMPs described herein can in aspects be designed to be administered via subdermal, intramuscular, or similar modes of injection, it is a particularly beneficial aspect of the invention that compositions comprising particles of substantially uniform size and/or shape, especially spherical/spheroid particles, can be contained in and delivered via relatively thin (and thus relatively painless) needle delivery systems, as exemplified by the microparticle-containing needle composition shown in FIG. 3, the PLGA monodisperse microparticles (microspheres) shown therein having an average diameter of 21 μm and shown in a dispersion within a 27 G needle.

As stated previously, compositions or formulations of the present invention can be administered by subcutaneous injection, intramuscular injection, or another parenteral route. According to specific embodiments, the composition(s) are delivered via a delivery system. The delivery system can comprise a means for administering the composition(s) of the present invention parenterally, such as by injection, further such as via a needle-free injection technology (NFIT), autoinjection device using a modified needle/injection system, or a traditional needle delivery system. An NFIT technology can comprise technology based on Lorentz forces, shock waves, pressure by gas, pressure by electrophoresis, or other technology capable of driving or propelling the compositions of the present invention through the skin without the use of a hypodermic needle. An autoinjection device can comprise a mechanical device which inserts a short needle and delivers composition to the target patient essentially simultaneously. Such a device can be useful for those with a needle phobia and can improve safety, assist in patient comfort and treatment protocol, and provide means for an increased number of patients to self-administer the compositions of the invention should use of a traditional hypodermic needle be problematic.

According to most common embodiments, the composition(s) or formulations of the present invention are administered via a hypodermic needle delivery system. A hypodermic needle injection system can be any suitable type of such a system. According to embodiments, the needle of such a system/composition is a 20-gauge needle or smaller, such as a 20-gauge, 21-gauge, 22-gauge, 23-gauge, 24-gauge, 25-gauge, or 26-gauge needle, such as a 27-gauge needle, a 28-gauge needle, or a 30-gauge needle. According to certain embodiments, the delivery system of the LARTCMP composition(s)/formulation(s) of the present invention can comprise a needle classified as smaller than 30-gauge. According to certain embodiments, the inner diameter of the needle is about 0.1 mm (100 μm)-about 0.61 mm (610 μm), for example about 0.1 mm (100 μm)-about 0.6 mm (600 μm), about 0.2 mm (200 μm)-about 0.5 mm (500 μm), or about 0.2 mm (200 μm)-about 0.4 mm (400 μm). In aspects the inner diameter of a needle of a delivery system is about 0.2 mm (200 μm)-about 04 mm (400 μm), such as about 0.210 mm (210 μm)-about 0.39 mm (390 μm). According to certain embodiments, the inner diameter of the needle included in the administration kit and used for injecting the LARTCMP compositions of the present invention can be no greater than about 0.5 mm (500 μm).

According to common embodiments, the delivery system for the composition(s) of the present invention comprises a 20-gauge hypodermic needle (or a needle equivalent or similar to such a needle) or smaller, the needle having an outer diameter of between about 0.3 mm (300 μm)-about 0.91 mm (910 μm), such as for example about 0.4 mm (400 μm)-about 0.8 mm (800 μm), or more specifically about 0.3 (300 μm)-about 0.8 mm (800 μm, as in about 0.4 mm (400 μm-about 0.7 mm (700 μm). In aspects, the outer diameter of the needle used for injecting the LARTCMP pharmaceutical compositions and/or formulations of the present invention can be no more than approximately 0.908 mm (908 μm). According to yet alternative embodiments, the outer diameter of the needle used for injecting the LARTCMP pharmaceutical compositions and/or formulations of the present invention can be no more than approximately 0.8 mm (800 μm).

In some aspects, the delivery needle for the compositions or formulations of the present invention has an inner diameter no larger than a 20-gauge needle (or a needle equivalent or similar to such a needle). Also or alternatively, the delivery needle can have an outer diameter no larger than a 20-gauge needle.

The needle of a delivery system or kit of the invention can be, e.g., a 20-gauge, 21-, 22-, 23-, 24-, 25-, 26-, 27-, 28-, 29-, or a 30-gauge needle or any needle smaller than a 30-gauge needle, especially where the microparticle composition has maximum size characteristics, uniform size characteristics and/or uniform shape characteristics, as exemplified above, such that the likelihood of injection failure due to agglomeration is very low. According to specific embodiments, the injection needle present in the kit has an inner diameter no greater than about 0.603 mm (603 μm), and/or also or alternatively has an outer diameter no greater than about 0.91 mm (910 μm) as described previously. In aspects, the injection needle present in the kit is of a typical size 20-gauge or smaller in inner diameter, outer diameter, or both. In some embodiments, the injection needle of the delivery system is classified as having a gauge which is equal to or smaller than that of a 23-gauge needle. In some embodiments, the injection needle of the delivery system is classified as having a gauge which is equal to or smaller than that of a 24-gauge needle. In some embodiments, the injection needle of the delivery system is classified as having a gauge which is equal to or smaller than that of a 25-gauge needle. In some embodiments, the injection needle of the delivery system is classified as having a gauge which is equal to or smaller than that of a 26-gauge needle. Needles typically will have an inner diameter to outer diameter ratio of between about 0.2-about 0.8, usually between about 0.45-about 0.7, and often between about 0.5-about 0.65 (e.g., 0.51-0.67 or 0.52-0.66).

Typically, a needle of a kit or needle-containing composition will have an inner diameter of about 0.6 mm or less, such as about 0.525 mm or less, and often about 0.45 mm or less. According to some embodiments, the needle has an inner diameter of about 0.4 mm (400 μm) or smaller. According to other aspects, the needle also or alternatively has an outer diameter of about 0.9 mm or less, about 0.85 mm or less, or about 0.8 mm (800 μm) or smaller (e.g., about 0.4-0.9 mm, such as about 0.5-0.85 mm, e.g., about 0.51 mm-about 0.82 mm).

In aspects, most if not substantially all of most, substantially all, or all of the particles of the composition in the present invention have a maximum diameter in any one direction of no more than approximately 120 μm, and usually no more than about 100 μm, such that an agglomeration of 3 particles provides a maximum diameter in any one direction of no more than approximately 360 μm, and usually no more than about 300 μm, and such a composition is usually delivered using a syringe having an inner diameter sufficiently capable of delivering the composition without fear of failure due to clogging by an agglomeration of 3 particles, that is having an inner diameter of at least approximately about 350 μm, e.g., about 400 μm or more. This principle can be applied to other maximum particle sizes with respect to other types of particles described in this disclosure.

According to an alternative illustrative embodiment, most if not substantially all of most, substantially all, or all of the particles of the composition in the present invention have a maximum diameter in any one direction of no more than approximately 90 μm, such that an agglomeration of 3 particles provides a maximum diameter in any one direction of no more than approximately 270 μm, and such a composition is delivered using a syringe having an inner diameter sufficiently capable of delivering the composition without fear of failure due to clogging by an agglomeration of 3 particles, that is having an inner diameter of at least approximately 270 μm, for example approximately at least about 270 μm, about 280 μm, about 290 μm, or at least about 300 μm.

According to another alternative exemplary embodiment, most if not substantially all of most, substantially all, or all of the particles of the composition in the present invention have a maximum diameter in any one direction of no more than approximately 80 μm, such that an agglomeration of 3 particles provides a maximum diameter in any one direction of no more than approximately 240 μm, and such a composition is delivered using a syringe having an inner diameter sufficiently capable of delivering the composition without fear of failure due to clogging by an agglomeration of 3 particles, that is having an inner diameter of at least approximately 240 μm, for example approximately at least about 240 μm, about 250 μm, about 260 μm, or at least about 270 μm.

### V. Kits/Systems

The pharmaceutical compositions and their constituent components, such as delivery MPCs in dried form, diluents, MPCs in reconstituted (liquid) form, and delivery systems can be provided as a “kit.” A “kit” in this sense means any collection of the various components of the inventive compositions described herein and one or more devices for storage, mixing, and/or administration of such compositions. The term “system” might alternatively be used to describe such collections. A kit can also comprise instructions for use and/or devices or materials for validating one or more aspects of a composition or of the kit components. Such a kit can comprise, e.g., one or more of the following elements: a) MPC in dried form contained within a first storage component or alternatively reconstituted (MPC in liquid form, having already been reconstituted with diluent) in a first storage component; b) diluent contained within a second storage component; c) a transfer mechanism for withdrawing diluent from the second storage component and adding it to the dried MPC of the first storage component; d) a delivery system prefilled with reconstituted (liquid) formulation or e) an empty delivery system.

The first storage component of a kit according to some aspects, containing an effective amount of an MPC in dried (e.g. cryodessicated) form or alternatively containing an effective amount of an MPC in reconstituted (liquid) form can be sealed with a seal capable of being penetrated by a material transfer mechanism, e.g. a needle. The storage component can be a vial, ampule, bottle, or similar such container made of glass or inert substance such as certain plastics which protects and does not react with the material contained therein. Such containers are commonly used in the art. A seal for the storage container can be made of a rubber, a silicone, polypropylene, PTFE, Teflon, or any similar material commonly used in the art to create a needle-penetrable seal in a vial or container cap. Such a seal can be comprised of a combination of any one or more such materials, such as a silicone/PTFE seal, a rubber/PTFE seal, or similar. According to further embodiments, the storage component containing an effective amount of the composition/formulation can be sealed with a seal capable of maintaining a sterile environment within the storage component. According to some embodiments, the seal is capable of preventing contamination.

According to some aspects a second storage component of a kit can comprise diluent for reconstitution of dried MPC when present in such a kit. Such a second storage component of a kit can be a vial, ampule, bottle, made of glass or inert substance, such as certain plastics which protects and does not react with the material contained therein as described for the first storage container. This second storage container can be similar to, the same as, or different from the first storage container in its design. Such a second storage container can be sealed according to the manner and with materials described for the first storage component so as to also prevent contamination. Such a second storage component can be designed to accept a transfer device designed to withdraw diluent contained therein so as to facilitate transfer of the diluent to the first storage container to reconstitute the dried material contained therein.

A kit can further comprise a transfer mechanism for withdrawing diluent from the second storage component and adding it to the dried MPC of the first storage component. Such a transfer device can be any transfer device capable of penetrating a cap or seal of the diluent storage container, withdrawing an amount of diluent contained therein, and adding the withdrawn diluent to a storage container containing dried MPC. According to common embodiments, such a transfer device is a syringe and needle system commonly used and would be commonly known in the art for such a transfer of materials. In certain embodiments, the transfer device can be the injection syringe of the delivery system, intended to withdraw diluent from a second storage component containing an appropriate amount of diluent (the diluent optionally containing one or more excipients), facilitate the addition of the diluent to the dried MPC, withdraw the reconstituted formulation, and administer the formulation to a recipient in need.

A kit can yet further comprise a delivery system for delivering a composition/formulation of the present invention to a live being. Such a delivery system can be any delivery system capable of administering an effective amount of composition/formulation of the present invention to a live being. In common embodiments, the delivery system is designed to administer the composition/formulation via injection, such as via subdermal or intramuscular injection. The delivery system can comprise an injection needle, needle system, or needleless delivery system capable of administering and delivering the microparticles of the present invention to a patient in need of treatment. According to a common aspect of the invention, the delivery system is an injection syringe comprising a needle delivery system. A composition/formulation of the present invention can be pre-filled in the syringe portion of the delivery system and as such be ready to be injected into the living being via an attached needle delivery system with the characteristics as described elsewhere herein. Alternatively, a second common aspect of the invention is a syringe and needle delivery system wherein the syringe is not pre-filled with composition/formulation, but wherein an attached needle system is capable of, in certain embodiments, penetrating a cap or seal of a first storage container containing the composition/formulation to be administered, the syringe is capable of facilitating the withdrawal of the composition/formulation from the storage container, and the system is then capable of administering the withdrawn composition/formulation to the live being.

The composition/formulation present in a kit can be conveniently presented in unit dosage form. Alternatively, they can be presented in multi-dose, or multi-unit form. According to certain embodiments, the composition(s)/formulation(s) described herein can be provided as part of a delivery system or packaged kit as previously described, such a kit comprising a single dose of the compositions and/or formulations of the present invention. Alternatively, such a kit can comprise multiple doses of the compositions and/or formulations of the present invention such that multiple doses can be administered over a predetermined time period, each single dose of the multiple dose series being derived from or originally housed within a single kit. For example, a single kit can comprise a single dose of a composition/formulation described herein. Alternatively a single kit can comprise one, two, three or more doses, such as four, five, or six doses or more, such that a single dose can be administered on day one, a second single dose can be administered on day two, a third single dose can be administered on day three and so on, or alternatively a single dose can be administered on day one, a second dose can be administered one week later, a third dose can be administered two weeks after the initial dose, and so on, or alternatively a single dose can be administered on day one, a second dose can be administered one month later, a third dose can be administered two months after the initial dose, and so on, or alternatively a single dose can be administered on day one, a second dose can be administered two, three, four, five, or six months after the initial dose and a second dose can be administered one, two, three, four, five, or six months after the second dose and so on. In such examples, each of the subsequent doses can originate from the same kit as the initial dose.

### 6. Therapeutic Methods and Other Methods of Use

In one aspect of the invention, the invention is a method for treating a treprostinil-treatable condition with the composition(s)/formulation(s) described herein, administering an effective amount of composition/formulation described herein to a subject in need thereof. A number of diseases and conditions can benefit from the administration of treprostinil. One common aspect of the invention is the method of treating pulmonary arterial hypertension (PAH) with the compositions and formulations described in this invention. According to some embodiments, an effective dose the composition(s)/formulation(s) described herein can be administered to a patient having been diagnosed with PAH utilizing the delivery systems and administration schedules described herein.

According to certain embodiments, the quantity of composition(s)/formulation(s) administered will vary depending on the patient and the mode of administration and can be any effective amount. In one embodiment, a typical dosage can include about 0.01 mg-about 3000 mg, for example about 0.01 mg-about 3000 mg, about 0.01 mg-about 2500 mg, about 0.01 mg-about 2000 mg, or approximately 0.01 mg-about 1500 mg. In another aspect, the dosage can include about 0.01-about 1000 mg, for example about 0.01 mg-about 1400 mg, about 0.01 mg-about 1300 mg, about 0.01 mg-about 1200 mg, or about 0.01 mg-about 1100 mg. In further embodiments, a typical dose can range from about 0.01 mg-about 100 mg, such as about 0.01 mg-about 1000 mg, 0.01 mg-about 900 mg, about 0.01 mg-about 800 mg, about 0.01 mg-about 700 mg, about 0.01 mg-about 600 mg, about 0.01-about 500 mg, about 0.01-about 400 mg, about 0.01-about 300 mg, or about 0.01-about 200 mg. In yet further aspects, a typical dose of LARTCMP composition administered can be about 0.01 mg-about 10 mg, such as about 0.01 mg-about 9 mg, about 0.01 mg-about 8 mg, about 0.01 mg-about 7 mg, about 0.01-about 6 mg, about 0.01-about 5 mg, about 0.01-about 5 mg, about 0.01 mg-about 5 mg, about 0.01 mg-about 4 mg, about 0.01 mg-about 3 mg, about 0.01-about 2 mg, or about 0.01-about 1 mg. In one aspect, the amount of treprostinil in the composition is about 50-200 mg, such as about 75-150 mg, such as about 80-120 mg, or about 100 mg. In one aspect, the ratio of drug to microparticle is also or alternatively about 2.5-25% or about 5-25%, such as about 7.5-17.5% or 7.5-15%, or about 7.5-12.5%, e.g., about 8-12%, or about 10%.

### I. Application in General/Treatment of PAH

In one embodiment, the method of treatment includes selecting a subject having been diagnosed with pulmonary arterial hypertension (PAH) or also or alternatively having demonstrated the clinical signs and symptoms of PAH such that treatment is determined by a trained healthcare provider to be appropriate and administering to the subject a therapeutically effective amount of composition/formulation described herein.

A subject in need of treatment can be a subject having been diagnosed with PAH based on, e.g., measurements of pulmonary artery pressure and RV systolic pressure in order to estimate pulmonary vascular resistance and also or alternatively to identify morphologic changes associated with pulmonary arterial hypertension, e.g. through the use of Doppler echocardiography, or other symptoms and/or diagnostic indicator(s). In alternative embodiments, technologies used alone or in conjunction with Doppler echocardiography can be used, such as pulmonary ventilation/perfusion scans (V/Q scans), pulmonary function tests, overnight oximetry, and serology tests for connective tissue disorders. In alternative or complimentary embodiments, right-heart catheterization can be used to confirm diagnoses and/or to evaluate the dynamics of patient blood flow. In certain embodiments, a mean pulmonary artery pressure (mPAP)>25 mmHg and pulmonary vascular resistance (PVR)>3 Wood units are the clinical diagnostic criteria used for diagnosing PAH. In certain embodiments, patients for whom idiopathic PAH is suspected, patients are identified through acute vasodilator response testing using inhaled nitic oxide or with intravenously administered epoprostenol or adenosine.

In certain embodiments, the method of treating pulmonary arterial hypertension (PAH) includes administering LARTCMPs comprising a therapeutically effective amount of a treprostinil compound, such as treprostinil, wherein the therapeutically effective amount can be from about 0.1 mg to about 2000 mg, for example about 0.1 mg to about 2000 mg, such as about 10 mg to about 1800 mg, as in about 50 mg to about 1500 mg, for example about 100 mg to about 1400 mg, about 200 mg to about 1300 mg, about 300 mg to about 1200 mg, about 400 mg to about 11 mg, such as about 500 mg to about 1000 mg.

In some aspects, the therapeutically effective amount of treprostinil or other treprostinil compound can be administered as a single dose in the performance of such a method. In aspects, the therapeutically effective amount of treprostinil, a prodrug, thereof, a hydrate thereof, a solvate thereof, a polymorph thereof, or a pharmaceutically acceptable salt thereof, can be administered as single doses separated in time by a period ranging between 1 day and 6 months. In aspects, the therapeutically effective amount of the treprostinil compound can be administered by injectable route or by other parenteral route. Unless otherwise indicated or clearly contradicted by context or plausibility, these principles will also be applicable to the administration of the inventive compositions in connection with the treatment or prevention, or enhancement of either thereof, of other treprostinil compound-treatable conditions.

### II. Alternative Therapeutic Targets

In another aspect, compositions of the invention are administered in an effective amount to a subject, such as a human patient, that has been diagnosed with a kidney function condition.

In one aspect, compositions of the invention are administered in an effective amount to treat a lung condition. Such a lung condition can include but not be limited to pulmonary fibrosis, chronic thromboembolic pulmonary hypertension, interstitial lung disease, or asthma.

According to yet further aspects of the present invention, the composition(s) and/or formulations described herein are utilized in the treatment of peripheral vascular disease, prevention of ischemic lesions, or neuropathic diabetic foot ulcer.

In other aspects, the compositions of the invention are administered to cause, promote, or enhance one or more physiological effects. In one exemplary aspect, the administration of the composition results in increased oxygen flow or vasodilation.

In another aspect, the composition(s)/formulation(s) of the present invention are used to treat gastric hypersecretion, gastric ulcers, or duodenal ulcers.

Inflammatory disorders offer yet another treatment target embodied by a further aspect of the present invention.

In still another embodiment, the method results in reduced pro-inflammatory cytokine expression of TNF-α, IL-1β or interferon gamma or upregulation/expression of IL-10. As such it is a further aspect of the present invention to treat inflammatory disorders with a composition/formulation described herein.

Compositions of the invention can be administered to increase blood perfusion and to treat conditions associated with low perfusion, such as ischemia. In another aspect, compositions of the invention are administered to treat, prevent, or enhance the treatment or prevent of a thrombosis-related disease or condition.

In yet another embodiment, the compositions of the invention are administered to treat or modulate one or more microcirculatory system disorders which are, e.g., affected by, induced or exacerbated when x-ray, NMR or ultrasonic contrast media are administered.

### III. Direct & Prophylactic Treprostinil Treatment

In aspects, treprostinil can be used as either a direct treatment for, or a prophylactic treatment for, any one of the aforementioned diseases or conditions or for any disease or condition which may benefit from the administration of treprostinil or a treprostinil compound.

For example, a composition/formulation as described herein can be administered in a therapeutically effective amount to a patient having been diagnosed with PAH, kidney function condition, lung condition such as pulmonary fibrosis, chronic thromboembolic pulmonary hypertension, and interstitial lung disease and asthma, peripheral vascular disease, prevention of ischemic lesions, neuropathic diabetic foot ulcers, gastric hypersecretion, gastric ulcers, or duodenal ulcers, inflammatory disorders, or microcirculatory system disorders when x-ray, NMR or ultrasonic contrast media are administered. In one embodiment a composition/formulation as described herein can be administered in a therapeutically effective amount to a patient having been diagnosed with microcirculatory system disorders when an MRI contrast agent is administered.

The compositions of the invention also can be administered to prevent such diseases or conditions. Prevention in this respect can be mean any reduction of the likelihood of occurrence, frequency of occurrence, or severity of occurrence of any such disease or condition.

The means of administration of treprostinil or treprostinil compound for any such treatment or prophylactic treatment can be via administration or application of any of the various compositions described herein. According to some embodiments, a LARTCMP composition/formulation of the present invention can be administered via parenteral administration. According to alternative embodiments, a LARTCMP composition/formulation of the present invention can be administered via subcutaneous administration; intravenous, intra-arterial, or intramuscular injection; intrapleural administration; intraperitoneal administration; intracavitary or intravesical instillation; or by intraocular, intraventricular, intralesional, or intraspinal administration. According to further embodiments, a LARTCMP composition/formulation of the present invention can be administered by application to mucous membranes, such as that of the nose, throat, and bronchial tubes. Also or alternatively, a LARTCMP composition/formulation of the present invention can be administered by any similar and effective mode of administration known in the art and suitable for the target disease state. According to specific embodiments, routes for administration can include via parenteral (including subcutaneous, intramuscular, intravenous, intradermal, intratracheal, and epidural) administration. Also or alternatively, routes of administration can be those suitable for dosage forms such as dry powder to be resuspended in a diluent prior to injection or as a ready-to-inject suspension or other dosage forms available to those skilled in the art.

### IV. Administration/Treatment Regimens

The treatment and preventative methods described above can be carried out using any suitable administration or treatment regimen.

According to one treatment protocol, the method of treatment can comprise treating personnel first identifying a subject in need of treatment with a formulation of the present invention; second administering to the subject a single dose of a therapeutically effective amount of a pharmaceutical composition/formulation as described herein; third monitoring the patient for reaction and response; and finally optionally administer to the subject one or more subsequent doses separated in time from one another from a period of between 1 day to about 6 months (e.g., about 1-30 days, about 1-60 days, about 1-90 days, about 1-100 days, about 1-120 days, or about 1-150 days).

In some aspects, the therapeutically effective treatment with a composition/formulation as described herein can be a single dose. Alternatively, the therapeutically effective treatment with a composition/formulation as described herein can be a series of single doses, e.g. a regimen of multiple doses separated in time from one another from a period of between 1 day to 6 months.

Such appropriate therapeutically effective treatment and dosing regimen(s) can be selected, adjusted or titrated by the clinician according to methods known to the clinician in order to obtain the optimal clinical response. According to primary embodiments, the amount of composition/formulation administered can be that amount which is therapeutically effective. The dosage to be administered can depend on the characteristics of the subject being treated, for example but not being limited to, the particular animal or human subject treated, age, weight, body mass index, body surface area, health, types of concurrent treatment, if any, and frequency of treatments, and can be easily determined by one of skill in the art, e.g. a clinician.

Diseases or conditions that can be treated by the practice of such methods can be any condition or disease that can be treated by the release of the treprostinil compound over the treatment period (which is usually a period of about 1-180 days). In one aspect, the disease to be treated is PAH. In another aspect, the disease or condition to be treated or prevented is a different disease or condition, numerous examples of which have been provided herein or are otherwise known in the art. In one aspect, the method is performed in order to detectably modify vasodilation in a subject or vasodilation of one or more systems in a subject, such as vasodilation of pulmonary and systemic arterial vascular beds. In another aspect, the method is performed to cause or promote platelet activation. The term “promote” in this respect can mean to increase the likelihood of an activity/effect occurring, to increase the extent of the activity/effect, or to increase the duration of the activity/effect. In other embodiments, the method is performed to increase the likelihood/enhance or cause smooth muscle cell proliferation. In still another aspect, the method is performed to treat or prevent dyspnea, fatigue, and low exercise capacity, or to enhance other treatments for these conditions. The methods generally can be combined with other known and suitable treatments for any of the diseases or conditions described herein, such as PAH. The methods can enhance the performance of other methods or together create a better overall treatment or prevention of the relevant disease or condition.

### V. Patient/Provider Satisfaction & Efficacy

Additional aspects of the invention are embodied in the treatment or prevention of any of the above-described diseases or conditions or modulation of the above-described physiological effects in association with one or more benefits that can be attributable to the LARTCMP element of the administered formulation or composition.

In one aspect, the employment of the methods of the invention is expected to be associated with a measurement improvement/enhancement in therapeutic, prophylactic, or physiological efficacy as determined by one or more clinical endpoints for the disease or condition to be treated, such as those endpoints associated with PAH products and trials. In one envisioned embodiment, the method of the invention results in an at least about 5% improvement, an at least about a 10% improvement, an at least about 15% improvement, an at least about a 20% improvement, or an at least about a 25% improvement, or even higher improvement in one or more endpoints or other efficacy markers. Examples of efficacy markers are described below.

In one aspect, the efficacy indicator that is improved in the method of the invention is demonstrated through an improvement in the 6 Minute Walking Distance Test as compared to other products available to treat PAH. The 6 Minute Walk Test is a test in which the distance a patient is able to walk over a total of six minutes on a hard, flat surface. Results, recorded at baseline (prior to treatment) and again after 12 weeks of therapy, are reported as the 6 Minute Walk Distance (6MWD) in meters. In an exemplary embodiment, performance of a method of the invention in PAH patients after 12 weeks of treatment results in an improvement from a baseline of about 5%, about 10%, about 15%, about 20%, or about 25% or more in the distance such a patient can walk in a 6MWD test.

Also or alternatively, treatment of pulmonary arterial hypertension (PAH) with a composition or formulation of the invention can be associated with a measurable improvement in the 1-year survival rate as compared with one or more currently FDA approved products (achieving, e.g., a 1-year survival rate of about 90% or higher, such as at least about 92% or at least about 95%). Also or alternatively, the methods of the invention can be associated with a 2-year survival rate that is better than one or more currently FDA approved products (e.g., achieving a survival rate of at least about 80%, such as at least about 82.5%, at least about 85%, at least about 87.5%, at least about 90%, at least about 92.5%, or at least about 95%). In other aspects, performance of such a method is associated with a measurable improvement in performance over one or all of the currently FDA approved products in terms of 3-year survival rate (e.g., achieving a 3-year survival rate of at least about 75%, at least about 85%, at least about 87.5%, or at least about 90%).

According to certain embodiments, treatment with the pharmaceutical composition(s) or formulation(s) of the present invention yields a lower reported rate of side effects and also or alternatively a measurably lower reported rate of adverse reactions than other existing and currently marketed injection- or infusion-administered PAH pharmaceuticals, such as REMODULIN®. According to certain embodiments, the administration of the composition(s)/formulation(s) described herein result in an average population treated for PAH having a measurably lower reported rate of any one or more side effects than that provided by treatment over the same length of time with REMODULIN®, such as, for example at least 0.5% lower, or at least about 1% lower, at least about 5% lower, or at least about 10% lower than that reported for treatment over the same length of time with REMODULIN®.

According to one embodiment, less than about 25% of patients treated with a composition or formulation of the present invention report headache as a side effect of treatment as determined by a sufficiently powered clinical study. Also or alternatively, according to certain embodiments, less than about 20% of patients treated with a composition or formulation of the present invention report nausea as a side effect as determined by a sufficiently powered clinical study. According to embodiments, less than about 25% of patients treated with a composition or formulation of the present invention also or alternatively report diarrhea as a side effect as determined by a sufficiently powered clinical study. In one aspect, less than about 15% of patients (e.g., less than about 10%) treated with a composition or formulation of the present invention also or alternatively report rash as a side effect of treatment as determined by a sufficiently powered clinical study. Also or alternatively, according to certain embodiments, less than about 10% of patients (e.g., less than about 9% of patients, less than about 7.5% of patients, or less than about 5% of patients) treated with a composition or formulation of the present invention report dizziness as a side effect of treatment as determined by a sufficiently powered clinical study. In other aspects, less than about 10% of patients (e.g., less than about 9% of patients or less than about 7.5% of patients) treated with a composition or formulation of the present invention report edema as a side effect of treatment as determined by a sufficiently powered clinical study. Also or alternatively, according to certain embodiments, less than about 8% of patients treated with a composition or formulation of the present invention report pruritis as a side effect of treatment as determined by a sufficiently powered clinical study (e.g., less than about 5% of such patients report such side effect). According to further embodiments, less than about 5% of patients (e.g., less than about 4% of patients, such as less than about 2.5% of patients) treated with a composition or formulation of the present invention also or alternatively report hypotension as a side effect of treatment as determined by a sufficiently powered clinical study.

According to certain embodiments, administration of the composition(s) and formulation(s) of the present invention results in patient compliance and patient/user satisfaction at least comparable with or greater than that associated with the currently FDA approved drugs comprising treprostinil compounds used to treat pulmonary arterial hypertension (PAH). Methods for determination of patient or user compliance or satisfaction are known in the art. patient or physician interview or formal, appropriately powered clinical study, or appropriately powered and controlled query of patients and physicians using the compositions by means such as a questionnaire. It is envisioned that an at least about 5% improvement, at least about 10% improvement, an at least about 15% improvement, or at least about a 20% improvement in patient or user (e.g., physician) satisfaction and/or patient compliance can be achieved by use of the compositions of the invention as compared to any or all of the currently approved FDA treatment methods.

### 8. Use of Alternative Means for Achieving Results/Performing Functions

The above-described aspects of the invention and elements thereof can be carried out by any suitable alternatives to any of the recited compositions, devices, and methods recited herein, especially when such aspects or elements are directed to subject matter that is known in the art. In this respect, the disclosure here can be considered to provide various “means” for carrying out aspects of the invention.

In one such exemplary aspect, the various carriers described herein can also be considered “carrier means” with respect to an aspect of the invention in which a treprostinil compound is described as comprising a “carrier means” or, e.g., “means for facilitating long acting release.” Such potential carrier materials can vary but serve to provide stability to the formulation, aid in the delivery the treprostinil compound, and/or to support the long acting release aspect of the formulation. The composition(s) can further optionally comprise one or more pharmaceutically acceptable excipients, which can also be described in terms of “means” for achieving certain results. The long acting release treprostinil compound particles of the composition(s)/formulation(s) of the present invention are present in an amount effective to treat a treprostinil-compound-treatable condition. Typically, the particles are suspended or dispersed in a liquid medium, by way of a diluent (or diluent means), which formulation composition can be in some aspects a viscous solution or a gel. In a further aspect, the invention provides compositions comprising storage and/or delivery devices comprising, packaged with, or otherwise associated with the particles or a formulation composition. Such delivery systems can also be described as means for injecting such compositions.

### EXEMPLARY ASPECTS

The following is a non-limiting list of exemplary aspects of the invention, which is intended to highlight some of the various embodiments of the invention.

1. A composition comprising biocompatible resorbable injectable sustaining-release treprostinil compound (BRIST) particles in an amount effective to treat a treprostinil-treatable condition over a treatment period of at least one day in a significant number of patients suffering from the condition, at least about 90% of the particles (a) having a maximum diameter of between about 5 μm and about 200 μm, (b) comprising an amount of the treprostinil compound sufficient to provide for an effective daily release over the treatment period, and (c) being configured to release the effective daily release over the treatment period.
2. A composition comprising biocompatible, non-resorbable, injectable, sustaining-release treprostinil compound (BNIST) particles in an amount effective to treat a treprostinil-treatable condition over a treatment period of at least one day in a significant number of patients suffering from the condition, at least about 90% of the particles (a) having a maximum diameter of between about 5 μm and about 200 μm, (b) comprising an amount of the treprostinil compound sufficient to provide for an effective daily release over the treatment period, (c) comprising an effective amount of a non-resorbable carrier material, and (d) being configured to release the effective daily release over the treatment period.
3. The composition of Aspect 2, wherein the particles are inert.
4. The composition of and one of Aspects 1-3, wherein the treprostinil compound comprises a prodrug of treprostinil.
5. The composition of any one of Aspects 1-3, wherein the treprostinil compound comprises a compound selected from an effective hydrate, solvate, polymorph, or salt of treprostinil.
6. The composition of any one of Aspects 1-3, wherein the treprostinil compound comprises an analog or derivative of treprostinil.
7. The composition of any one of Aspects 1-3, wherein the treprostinil compound comprises treprostinil in its typical form.
8. The composition of any one of Aspects 1-7, wherein the treprostinil compound comprises a mixture of two or more different treprostinil compounds.
9. The composition of any one of Aspects 1-8, wherein the composition comprises about 1 wt. % to about 80 wt. % of the treprostinil compound.
10. The composition of any one of Aspect 1 or 4-9, wherein the composition comprises about 70 wt. % to about 100 wt. % of the treprostinil compound.
11. The composition of any one of Aspects 1-10, wherein the composition comprises a total of between about 0.1 mg to about 2000 mg, such as about 0.1 mg to about 1000 mg, or about 500 mg to about 1000 mg of one or more treprostinil compounds.
12. The composition of any one of Aspects 1-11, wherein the composition comprises less than about 30% of a carrier component, such that at least about 70% of the particle is composed of the treprostinil compound.
13. The composition of Aspect 12, wherein the particles comprise less than about 10% of a carrier component.
14. The composition of Aspect 13, wherein the particles comprise less than about 5% of a carrier component.
15. The composition of Aspect 14, wherein the composition of the particles comprises less than about 1% of a carrier component.
16. The composition of Aspect 15, wherein at least 99% of the composition of the particles is composed of one or more treprostinil compounds.
17. The composition of Aspect 16, wherein at least 99.5% of the composition of the particles is composed of one or more treprostinil compounds.
18. The composition of any one of Aspects 1 or 4-17, wherein the particles comprise no carrier component.
19. The composition of any one of Aspects 1-11, wherein the composition comprises a carrier material that is biocompatible and that is present in an amount and configured to detectably facilitate the release of the treprostinil compound during the treatment period.
20. The composition of Aspect 19, wherein at least about 30% of the composition of the particles is composed of the carrier.
21. The composition of Aspect 20, wherein at least about 50% of the composition of the particles is composed of the carrier.
22. The composition of Aspect 21, wherein at least about 70% of the composition of the particles is composed of the carrier.
23. The composition of any one of Aspects 19-22, wherein the composition comprises a carrier material that is biocompatible and resorbable and that is present in an amount and configured to detectably facilitate the release of the treprostinil compound during the treatment period.
24. The composition of Aspect 23, wherein at least about 30% of the composition of the particles is composed of the carrier.
25. The composition of Aspect 24, wherein at least about 50% of the composition of the particles is composed of the carrier.
26. The composition of Aspect 25, wherein at least about 70% of the composition of the particles is composed of the carrier.
27. The composition according to any one of Aspects 19-26, wherein the carrier material is bioactive, forming detectably measurable bonds with hard tissues, soft tissues, or both hard and soft tissues.
28. The composition of Aspect 27, wherein at least 50% of the composition of the carrier material is formed of a bioactive composition.
29. The composition of any one of Aspects 23-26, wherein the carrier material comprises a resorbable bioceramic carrier material.
30. The composition of Aspect 29, wherein at least about 50% of the carrier material is a resorbable bioceramic material.
31. The composition of Aspect 30, wherein at least about 90% of the carrier material is a resorbable bioceramic material.
32. The composition of Aspect 31, wherein at least about 98% of the carrier material is a resorbable bioceramic material.
33. The composition according to any one of Aspects 29-32, wherein the carrier material consists of two or more resorbable bioceramic compounds.
34. The composition of Aspect 33, wherein the bioactive composition is a hydroxyapatite compound.
35. The composition of Aspect 34, wherein the hydroxyapatite material is sintered hydroxyapatite, synthetic hydroxyapatite, or synthetic sintered hydroxyapatite.
36. The composition of any one of Aspects 29-33, wherein the one or more resorbable bioceramic carrier materials comprises a non-crystalline bioceramic material.
37. The composition of Aspect 36, wherein the non-crystalline bioceramic material comprises a bioglass.
38. The composition of any one of Aspects 29-33, wherein the bioceramic carrier material is at least 50% composed of a single crystal material.
39. The composition according to any one of Aspects 29-33, wherein the bioceramic carrier material is at least 50% composed of a polycrystalline material.
40. The composition according to any one of Aspects 29-33, wherein the resorbable bioceramic composition is tricalcium phosphate.
41. The composition of any one of Aspects 29-33, wherein at least 50% of the bioceramic carrier material is composed of a calcium phosphate material.
42. The composition of Aspect 41, wherein the calcium phosphate material is selected from biphasic calcium phosphate (BCP), octacalcium phosphate (OCP), a di-calcium phosphate (DCP), or a monocalcium phosphate (MCP).
43. The composition of any one of Aspects 29-33, wherein at least 50% of the bioceramic carrier material is composed of a calcium carbonate or a calcium sulfate.
44. The composition according to any one of Aspects 23-26, wherein the carrier material comprises a proteinaceous material.
45. The composition of Aspect 44, wherein the proteinaceous material composes at least 50% of the carrier material.
46. The composition of any one of Aspects 44 or Aspect 45, wherein the proteinaceous material is bone.
47. The composition of any one of Aspects 44 or 45, wherein the proteinaceous material is collagen.
48. The composition of according to any one of Aspects 23-26, wherein the composition is extracellular matrix.
49. The composition of any one of Aspects 23-26, wherein the carrier material comprises a biodegradable polymer.
50. The composition of Aspect 49, wherein at least about 50% of the composition of the carrier material is composed of a biodegradable polymer.
51. The composition of any one of Aspect 49 or Aspect 50, wherein the biodegradable polymer exhibits surface-erosion rather than bulk erosion.
52. The composition of any one of Aspects 49-51, wherein the biodegradable polymer is a polyanhydride polymer.
53. The composition of Aspect 52, wherein the polyanhydride is sebacic acid (SA), p-carboxyphenoxyproane (CPP), or a combination thereof.
54. The composition of Aspect 52, wherein the polyanhydride is a polyanhydrids-co-imide.
55. The composition of any one of Aspects 49-51 wherein the composition is a poly(ortho) ester.
56. The composition of Aspect 55, wherein the poly(ortho)ester is characterizable as a type IV poly(ortho)ester or a self-catalyzed poly(ortho)ester.
57. The composition of any one of Aspect 49 or Aspect 50, wherein the biodegradable polymer comprises one or more resorbable polyester polymer (RPP) materials.
58. The composition of Aspect 57, wherein the resorbable polyester polymer material is composed of a polyglycolide (a PGA), a polylactic acid (a PLA), a polycaprolactone (a PCL), a polyhydroxybutyrate (a PHB), a copolymer of two or more thereof, or a mixture of any two or more thereof.
59. The composition of Aspect 58, wherein the resorbable polyester polymer material comprises poly(lactic-co-glycolic acid) (PLGA).
60. The composition of Aspect 59, wherein the PLGA is primarily poly-L-lactic acid (PLLA) (crystalline).
61. The composition of Aspect 59, wherein the PLGA is primarily poly-D-lactic acid (PDLA) (amorphous).
62. The composition of any one of Aspects 59-61, wherein the PLGA is a mixture of PLLA and PDLA.
63. The composition of Aspect 1, wherein the BRIST particles comprise a carrier material primarily comprising a copolymer selected from the group consisting of a copolymer of DL-lactide or copolymer of DL-lactide and glycolide.
64. The composition of Aspect 58, wherein the PLGA is a copolymer of DL-lactide and glycolide or a copolymer of DL-lactide.
65. The composition according to any one of Aspects 59-64, wherein the PLGA polymer has a monomer ratio between 50-100.
66. The composition according to any one of Aspects 57-65, wherein each of the one or more resorbable polyester polymer(s) has a molecular weight between 5,000-250,000 Daltons.
67. The composition according to any one of Aspects 18-66, wherein the particles comprise a carrier material that is composed of a mixture of different carrier materials.
68. The composition according to Aspect 67, wherein the mixture is selected from one or more biodegradable polymers and one or more proteinaceous materials.
69. The composition according to Aspect 67, wherein the mixture is selected from one or more biodegradable polymers and one or more resorbable bioceramics.
70. The composition according to Aspect 67, wherein the mixture is selected from one or more proteinaceous materials and one or more resorbable bioceramics.
71. The composition according to any one of Aspects 18-70, wherein the particular application of the active agent(s) will define the appropriate carrier formulation.
72. The composition according to any one of Aspects 23-71, wherein at least 90% the particles of the composition are resorbable within 6 months of administration.
73. The composition of Aspect 72, wherein at least 95% of the particles of the composition are resorbable within 6 months post administration.
74. The composition of Aspect 73, wherein at least 99% of the particles of the composition are resorbable within 6 months post administration.
75. The composition of Aspect 74, wherein no detectible amount of the particles of the composition remains after 6 months post administration.
76. The composition according to any one of Aspects 23-71, wherein at least 90% of the particles of the composition are resorbable within 3 months post administration.
77. The composition of Aspect 76, wherein at least 95% of the particles of the composition are resorbable within 3 months post administration.
78. The composition of Aspect 77, wherein at least 99% of the particles of the composition are resorbable within 3 months post administration.
79. The composition of Aspect 78, wherein no detectible amount of the particles of the composition remains after 3 months post administration.
80. The composition according to any one of Aspects 23-71, wherein at least 90% of the particles of the composition are resorbable within 1 week post administration.
81. The composition of Aspect 80, wherein at least 95% of the particles of the composition are resorbable within 1 week post administration.
82. The composition of Aspect 81, wherein at least 99% of the particles of the composition are resorbable within 1 week post administration.
83. The composition of Aspect 82, wherein no detectible amount of the particles of the composition remains after 1 week post administration.
84. The composition according to any one of Aspects 23-71, wherein at least 90% of the particles of the composition are resorbable within 1 day post administration.
85. The composition of Aspect 84, wherein at least 95% of the particles of the composition are resorbable within 1 day post administration.
86. The composition of Aspect 85, wherein at least 99% of the particles of the composition are resorbable within 1 day post administration.
87. The composition of Aspect 86, wherein no detectible amount of the particles of the composition remains after 1 day of administration.
88. The composition of any one of Aspects 19-22 wherein the particles are biocompatible and non-resorbable.
89. The composition of Aspect 88, wherein the particles are bioactive, forming detectably measurable bonds with hard tissues, soft tissues, or both hard and soft tissues.
90. The composition of Aspect 89, wherein at least 50% of the composition of the carrier material is formed of a bioactive composition.
91. The composition of Aspect 88, wherein the carrier material comprises a non-resorbable bioceramic carrier material.
92. The composition of Aspect 91, wherein at least about 50% of the carrier material is a non-resorbable bioceramic material.
93. The composition of Aspect 92, wherein at least about 90% of the carrier material is a non-resorbable bioceramic material.
94. The composition of Aspect 93, wherein at least about 98% of the carrier material is a non-resorbable bioceramic material.
95. The composition of any one of Aspects 91-94, wherein the bioactive composition is a hydroxyapatite compound.
96. The composition of Aspect 95, wherein the hydroxyapatite material is sintered hydroxyapatite, synthetic hydroxyapatite, or synthetic sintered hydroxyapatite.
97. The composition of any one of Aspects 88-96, wherein the carrier material consists of two or more non-resorbable bioceramic compounds.
98. The composition of any one of Aspects 88-97, wherein the particles comprise a carrier material that is composed of a mixture of different carrier materials.
99. The composition of Aspect 98, wherein the mixture of different carrier materials is selected from non-resorbable polymers and ceramics.
100. The composition of any one of Aspects 88-99 wherein the particular application of the active agent(s) will define the appropriate carrier material formulation.
101. The composition according to any one of Aspects 19-100, wherein the drug load of the carrier material is between the range of 1 to 80 wt %.
102. The composition according to Aspect 101, wherein the drug load of the carrier material is greater than 50 wt %.
103. The composition of any one of Aspects 1-102, wherein the particles have a size distribution such that at least 65% of the particles of the composition have a maximum particle diameter that is within 35% of the average particle diameter of the particles in the composition.
104. The composition of Aspect 103, wherein the particles have a size distribution such that at least 70% of the particles of the composition have a maximum particle diameter that is within 35% of the average particle diameter of the particles in the composition.
105. The composition of Aspect 104, wherein the particles have a size distribution such that at least 33% of the particles of the composition have a maximum particle diameter that is within 15% of the average particle diameter of the particles in the composition.
106. The composition of Aspect 105, wherein the particles have a size distribution such that at least 40% of the particles of the composition have a maximum particle diameter that is within 20% of the average particle diameter of the particles in the composition.
107. The composition of Aspect 106, wherein less than about 1% of the particles have a maximum diameter that is more than 66% greater than the average diameter of particles in the composition or less than about 66% less than the average diameter of particles in the composition.
108. The composition of Aspect 107, wherein less than about 10% of the particles have a maximum diameter that is more than 50% greater than the average diameter of particles in the composition.
109. The composition of Aspect 108, wherein less than about 10% of the particles have a maximum diameter that is more than 50% greater or less than about 50% less than the average diameter of particles in the composition.
110. The composition of any one of Aspects 1-109, wherein less than about 5% of the particles have a size that is below 5 μm.
111. The composition of any one of Aspects 1-110, wherein less than about 5% of the particles have a maximum diameter that is greater than 100 μm.
112. The composition of Aspect 110 or Aspect 111, wherein less than 7.5% of the particles have a size that is greater than 50 μm.
113. The composition of any one of Aspects 1-112, wherein at least 93% of the particles in the composition are between 5 μm and 200 μm in size.
114. The composition of any one of Aspects 1-113, wherein at least 65% of the particles in the composition have an average diameter in the range of 20 μm-150 μm.
115. The composition of Aspect 114, wherein at least about 70% of the treprostinil-loaded micro- or nanoparticles of the composition have a maximum diameter between 50-130 μm in size.
116. The composition according to any one of Aspects 1-115, wherein the maximum average dimension of three agglomerated particles of the composition is less than 500 μm.
117. The composition of Aspect 116, wherein the maximum average dimension of three agglomerated particles of the composition is less than 360 μm.
118. The composition of Aspect 116 or Aspect 117 wherein the frequency of agglomeration is at least 5% less than the frequency of agglomeration using a typical drug-loaded micro- or nanoparticle delivery system having:
a. a maximum particle size diameter which is at least 70% greater than the average particle size diameter;
b. a minimum particle size diameter which is at least 70% less than the average particle size diameter;
c. the maximum variation in any two or more directions of the particle size diameter varies by more than 70%; or
d. any combination of (a), (b), and/or (c) above.
   119. The composition according to any one of Aspects 1-118, wherein the diameter of at least 80% of the particles of the composition varies by no more than 15% in any direction.
   120. The composition of Aspect 119, wherein the diameter of at least 90% of the particles of the composition varies by no more than about 5% in any direction.
   121. The composition of Aspect 120, wherein the diameter of at least 80% of the particles of the composition varies by no more than about 2% in any direction.
   122. The composition of Aspect 121, wherein the diameter of at least 85% of the particles of the composition varies by no more than about 1% in any direction.
   123. The composition of Aspect 122, wherein the particles of the composition are substantially spherical.
   124. A formulation comprising any one of the compositions of Aspects 1-123, wherein the composition is in dry powder form and adapted for suspension with a reconstitution diluent to form a suspension of the particles.
   125. The formulation according to Aspect 124, wherein the formulation further contains one or more excipients.
   126. The formulation of Aspect 125, wherein the dried powder formulation comprises one or more excipients that when reconstituted form solvents in solution but are not part of the particles in suspension.
   127. The formulation of Aspect 126, wherein the reconstitution diluent contains one or more emulsion-forming materials.
   128. The formulation of Aspect 127, wherein the emulsion-forming material comprises one or more oils.
   129. The formulation of Aspect 128, wherein the one or more oils is/are selected from the group consisting of petroleum, animal, vegetable, plant, or oils of synthetic origin.
   130. The formulation of Aspect 129, wherein the formulation resulting from the reconstitution of the composition in dry powder form with the one or more oils forms an emulsion.
   131. The formulation of any one of Aspects 124-126, wherein the reconstitution diluent comprises water for injection.
   132. The formulation of Aspect 131, wherein the formulation resulting from the reconstitution of the composition in dry powder form with water for injection forms an aqueous solution.
   133. A formulation comprising any one of the compositions according to any one of Aspects 1-132, wherein the formulation is delivered using a delivery system.
   134. The formulation of Aspect 133, wherein the delivery system is a needle.
   135. The formulation of Aspect 134, wherein the needle of the delivery system has an inner diameter range of between 0.1 mm and 0.8 mm (100-800 μM).
   136. The formulation of Aspect 135, wherein the needle of the delivery system has an inner diameter no greater than 0.65 mm.
   137. The formulation according to any one of Aspects 134-136, wherein the needle of the delivery system has an outer diameter range of between 0.3 mm and 0.9 mm.
   138. The formulation of Aspect 137, wherein the needle of the delivery system has an outer diameter no greater than 0.85 mm.
   139. The formulation according to any one of Aspects 134-138, wherein the needle of the delivery system is classified as having a gauge which is equal to or smaller than 23-gauge.
   140. The formulation of Aspect 139, wherein the needle of the delivery system is classified as having a gauge which is equal to or smaller than 24-gauge or 25 gauge.
   141. The formulation of Aspect 140 or Aspect 139, wherein the needle has a ratio of inner diameter to outer diameter of about 0.4 to about 0.8.
   142. A delivery system packaged as a kit for administration of the long acting release pharmaceutical composition comprising treprostinil of any one of Aspects 1-141, wherein the kit comprises:
a. a storage component containing an effective amount of the sustaining-release pharmaceutical composition comprising treprostinil with or without resorbable polyester polymer carrier particles and with or without one or more adjuvants or excipients;
b. a needle configured to deliver the composition to a patient in need of receiving the pharmaceutical composition;
c. a syringe, either:
   i. prefilled with diluent, the diluent optionally containing one or more adjuvants or excipients or
   ii. capable of withdrawing, when used with a second needle optionally part of the kit, diluent which may or may not contain one or more adjuvants or excipients, from a second storage component containing such diluent, and transferring the diluent to the storage component containing an effective amount of the pharmaceutical composition comprising particles treprostinil, the composition optionally comprising carrier material.
      143. The delivery system of Aspect 142, wherein the storage component containing an effective amount of the long acting release treprostinil compound pharmaceutical composition comprising treprostinil with or without carrier material is sealed with a seal capable of being penetrated by a needle.
      144. The delivery system of Aspect 143 wherein the seal is capable of preserving a sterile environment.
      145. The delivery system of any one of Aspects 142-144, wherein the long acting release pharmaceutical composition comprising treprostinil compound with or without carrier material is in cryodesiccated form.
      146. The delivery system of any one of Aspects 142-145, wherein the long acting release pharmaceutical composition comprising treprostinil with or without carrier particles in cryodessicated form, with or without one or more adjuvants or excipients, is reconstituted with diluent either pre-filled in the syringe of the delivery system or stored in a second storage container of the kit, such diluent optionally comprising one or more adjuvants or excipients.
      147. The delivery system of any one of Aspects 142-146, wherein the needle for injection of the long acting release pharmaceutical composition comprising treprostinil is a 23-gauge needle or smaller.
      148. The delivery system of Aspect 147, wherein the needle for injection of the long acting release treprostinil compound composition has an inner diameter smaller than 0.4 mm (400 μM).
      149. The delivery system of any one of Aspects 147-148, wherein the needle for injection of the LAR pharmaceutical composition has an outer diameter smaller than 0.825 mm (825 μM).
      150. The delivery system of any one of Aspects 142-149, wherein the amount of treprostinil, the prodrug thereof, the hydrate thereof, the solvate thereof, the polymorph thereof, or the pharmaceutically acceptable salt thereof in the pharmaceutical composition in the delivery system packaged as a kit is from about 0.1 mg to about 2000 mg.
      151. The delivery system of any one of Aspects 142-150, wherein the amount of diluent optionally containing one or more adjuvants or excipients within the delivery system packaged as a kit is between 0.5 and 10 mL.
      152. The composition, formulation, or kit as described in any one of aspects 1-151, wherein the composition, formulation, or kit does not comprise an aerosol or an element in aerosol form.
      153. The composition, formulation, or kit as described in any one of aspects 1-152, wherein the composition or formulation is not delivered by infusion.
      154. The composition, formulation, or kit as described in any one of aspects 1-153, wherein the composition or formulation is not delivered by intravenous (IV) delivery.
      155. The composition, formulation, or kit as described in any one of aspects 1-154, wherein the composition or formulation is not delivered by inhalation.
      156. The composition, formulation, or kit as described in any one of aspects 1-155, wherein the composition or formulation is not administered more frequently than once per day.
      157. The composition, formulation, or kit as described in any one of aspects 1-156, wherein the composition, formulation, or kit does not comprise a hydrogel.
      158. The composition, formulation, or kit as described in any one of aspects 1-157, wherein the composition, formulation, or kit does not comprise a gel or an element in gel form.
      159. The composition, formulation, or kit as described in any one of aspects 1-158, wherein the composition, formulation, or kit does not comprise, a tablet or an element in tablet form.
      160. A method of treating, preventing, or modulating a condition in a mammalian subject, comprising administering to a patient having a treprostinil-treatable condition an effective amount of a composition according to any one of Aspects 1-159 to the subject.
      161. The method of Aspect 160, wherein the composition is administered as a single dose.
      162. The method according to any one of Aspect 160 or Aspect 161, wherein the composition is administered as a series of single doses, spread apart in time by about 1 day to about 6 months.
      163. The method of Aspect 162, wherein the composition is administered as a series of single doses spread apart in time by about 1 day to 3 months.
      164. The method of Aspect 163, wherein the composition is administered as a series of single doses spread apart in time by about 1 day to 1 week.
      165. The method of Aspect 164, wherein the composition is administered as a series of single doses spread apart in time by about 1 day.
      166. The method according to any one of Aspects 160-165 wherein the composition is administered through a parenteral route.
      167. The method according to Aspect 166, wherein the composition is administered by subcutaneous injection.
      168. The method according to Aspect 166, wherein the composition is administered by intramuscular injection.
      169. The method according to Aspect 166, wherein the composition is administered by intradermal injection.
      170. The method according to Aspect 166, wherein the composition is administered by epidural injection.
      171. The method according to any one of Aspects 160-170, wherein the condition is selected from the group comprising compromised kidney function, pulmonary fibrosis, peripheral vascular disease, ischemic lesions, neuropathic diabetic foot ulcers, chronic thromboembolic pulmonary hypertension, gastric hypersecretion, gastric ulcers, duodenal ulcers, inflammatory disorders, or microcirculatory system disorders induced or exacerbated when x-ray, NMR, MRI, or ultrasonic contrast media are administered.
      172. The method according to any one of Aspects 160-170, wherein the condition is pulmonary arterial hypertension (PAH).
      173. The method according to any one of Aspects 160-170, wherein the effective amount of the composition is provided prophylactically.
      174. A method of treating a treprostinil-treatable condition comprising administering to a patient having a treprostinil-treatable condition an effective amount of a composition according to any one of aspects 1-173, wherein the rate of compliance is equal to or greater than that obtained by FDA approved drugs comprising one or more of treprostinil or a treprostinil compounds used to treat pulmonary arterial hypertension (PAH) as determined by an appropriately powered clinical study.
      175. A method of treating a treprostinil-treatable condition comprising administering to a patient having a treprostinil-treatable condition an effective amount of a composition according to any one of aspects 1-173, wherein the rate of patient satisfaction is equal to or greater than that obtained by FDA approved drugs comprising one or more of treprostinil or a treprostinil compound used to treat pulmonary arterial hypertension (PAH) as determined by an appropriately powered and controlled study.
      176. The method of any one of Aspects 171-175, wherein the administration of the composition results in an improvement in an efficacy indicator test of greater than 1% over REMODULIN® when evaluated over a 12 week period.
      177. The method of any one of Aspects 171-176, wherein the administration of the composition results in an improvement in an efficacy indicator test of greater than 1% over ORENITRAM® when evaluated over a 12 week period.
      178. The method of any one of Aspects 171-177, wherein the administration of the composition results in an improvement in an efficacy indicator test of greater than 1% over TYVASO® when evaluated over a 12 week period.
      179. The composition according to any one of Aspects 1-178, wherein administration of the composition results in a lower reported rate of any one or more side effects than that reported by REMODULIN®, TYVASO®, ORENITRAM®, or a combination of two or all thereof, as determined by an appropriately powered clinical study.
      180. A composition comprising an effective amount of long acting release treprostinil compound microparticles and comprising a carrier means for enhancing the release of the treprostinil compound in the body of a mammalian subject following administration, and vehicle means for diluting the microparticles and providing for effective delivery of the microparticles through a delivery device or delivery means.
      181. A composition comprising a composition according to Aspect 180 and a delivery means for delivering the composition into the body of a mammalian recipient.
      182. The composition of Aspect 180 or Aspect 181 further comprising an excipient means for detectably enhancing one or more non-pharmacologic aspects of the composition.

1. A pharmaceutically acceptable composition comprising an effective amount of biocompatible, injectable, sustained-release treprostinil compound particles, at least about 90% of the particles having a maximum diameter of between about 10 μm and about 200 μm; at least 65% of the particles having a maximum particle diameter within 35% of the average particle diameter of the particles; and the particles releasing an effective amount of treprostinil over a treatment period of at least one day when administered to patients.

2. The composition of claim 1, wherein less than about 10% of particles of the composition have a maximum diameter that is more than 50% greater than the average diameter of particles in the composition.

3. The composition of claim 2, wherein the treprostinil compound is selected from the group comprising a) treprostinil; b) an analog or derivative of treprostinil; c) a prodrug, hydrate, solvate, polymorph, or salt of either of a) or b); or d) a mixture of any or all of (a), (b), or (c).

4. The composition of claim 3, wherein the composition does not comprise a carrier.

5. The composition of claim 3, wherein the particles of the composition comprise a carrier, and the drug load of the particles is between 1-80%.

6. The composition of claim 5, wherein the carrier is a non-resorbable carrier.

7. The composition of claim 5, wherein the carrier is a resorbable carrier.

8. The composition of claim 7, wherein the particles comprise a resorbable polyester polymer material carrier material at least primarily composed of a polyglycolide (a PGA), a polylactic acid (a PLA), a polycaprolactone (a PCL), a polyhydroxybutyrate (a PHB), a copolymer of two or more thereof, or a mixture of any two or more thereof.

9. The composition of claim 8, wherein, on average, at least 90% of particles are resorbable within 6 months of administration of the composition to patients.

10. The composition of claim 9, wherein the monomer ratio of the polyester polymer is between 50 and 100 and the molecular weight of the polyester polymer is between 10-200 kDa.

11. The composition of claim 10, wherein, on average, there is no detectible amount of particles remaining in a patient 3 months or more post-administration of the composition to patients.

12. The composition of claim 3, wherein the composition is in dry powder form and is adapted to form a suspension with a reconstitution diluent.

13. The composition of claim 3, the composition further comprising a needle for injecting the composition into patients, the needle having an inner diameter of less than 0.65 mm, an outer diameter of no greater than 0.85 mm, or both.

14. A method of treating a treprostinil-treatable condition in a patient comprising administering an effective amount of the composition of claim 1 to the patient.

15. The method of claim 14, wherein the composition is not delivered to the patient more than once per day.

16. The method of claim 15, wherein the composition is a composition according to claim 2.

17. The method of claim 16, wherein the composition is a composition according to claim 3.

18. The method of claim 16, wherein the composition is a composition according to claim 8.

19. The method of claim 18, wherein the condition is pulmonary arterial hypertension.

20. The method of claim 19, wherein the method comprises injecting the composition into the patients with a needle, the needle having an inner diameter of less than 0.65 mm, an outer diameter of no greater than 0.85 mm, or both.
